(19) United States
(12) Patent Application Publication
(10) Pub. No.: US 2026/0325353 A1
(43) Pub. Date: Oct. 22, 2026

(54) LASER SYSTEM HAVING A NON-PERPENDICULAR DEFLECTION ANGLE

(71) Applicant: TECHNOLAS PERFECT VISION GMBH, München (DE)

(72) Inventors: Wolfgang KÜGEL, München (DE); Christof DONITZKY, München (DE); Georg HENNIG, München (DE); Christian WÜLLNER, München (DE); Stefan BERGER, München (DE)

(21) Appl. No.: 19/653,865

(22) Filed: Apr. 21, 2026

(30) Foreign Application Priority Data
Apr. 22, 2025 (EP) 25171789.8

(51) Int. Cl. A61F 9/008

(52) U.S. Cl. CPC A61F 9/00804; A61F 9/00814; A61F 2009/00844; A61F 2009/00851; A61F 2009/00897

(57) ABSTRACT

The present disclosure relates to an ophthalmic laser system configured to perform laser treatments on an eye of a patient using a primary beam comprising a treatment laser beam, the ophthalmic laser system comprising a plurality of components including: a beam delivery system for focusing the primary beam so that the primary beam travels toward the eye to be treated, a scanning system for scanning a focus of the primary beam within the eye or on an anterior surface of the eye in two or more dimensions, wherein the beam delivery system comprises a stationary deflection element adapted to deflect the primary beam at a deflection angle toward the eye, wherein the deflection angle is defined as an angle between an incident portion of the primary beam along a first optical axis of one or more upstream optical elements of the beam delivery system, wherein the upstream optical elements are arranged in a beam path of the primary beam upstream of the stationary deflection element; and a deflected portion of the primary beam along a second optical axis of one or more downstream optical elements of the beam delivery system, wherein the downstream optical elements are arranged in the beam path of the primary beam downstream of the stationary deflection element; wherein the deflection angle is larger than 90°.

### TECHNICAL FIELD

The present invention relates to an ophthalmic laser system configured to perform laser treatments on an eye of a patient, such as treatments which form incisions within the cornea or the natural lens of the eye and a corresponding method for operating an ophthalmic laser system. The present invention also relates to systems for ablating a surface portion of the cornea, such as photorefractive keratectomy (PRK) and laser in situ keratomileusis (LASIK) treatments.

### BACKGROUND

Cataract surgery is one of the most common ophthalmic surgical procedures performed. The primary goal of cataract surgery is the removal of the aging lens and replacement with an artificial lens or intraocular lens (IOL) that restores some of the optical properties of the aged lens.

The major steps in cataract surgery consist of forming a corneal incision to allow access to the anterior chamber of the eye and incisions to correct for astigmatism (Limbal relaxing incisions, abbreviated as LRIs or astigmatic keratotomy, abbreviated as AK), cutting and opening the capsule of the lens to gain access to the lens (capsulotomy), lens fragmentation and removing the fragmented lens through phacoemulsification, irrigation and aspiration and in most cases placing an artificial intraocular lens in the eye.

Therefore, femtosecond laser-assisted cataract surgery is a multi-stage process which includes femtosecond laser surgery (LRI, AK, corneal incision, capsulotomy and lens fragmentation), a phacoemulsification process (emulsifying, liquifying and aspirating the lens) and manual surgical procedures (implantation of the intraocular lens).

Nowadays, space constraints in the arrangement of the laser systems in particular with respect to a patient often impede a proper handling of the femtosecond laser systems during operation. The requirement to safely deliver one or more beams to a target region, e.g. an eye of the patient, is particularly challenging.

Moreover, in order to reduce footprint and costs, it is desirable that femtosecond laser systems, which are configured for cataract surgery could also be used for forming corneal flaps for laser in situ keratomileusis (LASIK). A femtosecond laser for forming LASIK flaps, however, should be located close to an excimer laser system in order to make it easy for the patient to move from one system to the other. On the other hand, a femtosecond laser for cataract surgery should be located in the sterile operating theatre, where phacoemulsification and IOL implantation are carried out.

In view of the above, there is a need to provide a laser system which allows an efficient workflow in both use scenarios.

### SUMMARY

The above noted problems are at least partially addressed by the ophthalmic laser systems according to one of claims 1, 16 or 17 and the methods for operating an ophthalmic laser system according to one of claims 18, 19 or 20. Preferred embodiments thereof are accordingly set forth in the respective dependent claims.

The present invention relates to an ophthalmic laser system configured to perform laser treatments on an eye of a patient. The ophthalmic laser system uses a primary beam comprising a treatment laser beam. The ophthalmic laser system comprises a plurality of components including a beam delivery system for focusing the primary beam so that the primary beam travels toward the eye to be treated. The ophthalmic laser system further comprises a scanning system for scanning a focus of the primary beam within the eye or on an anterior surface of the eye in two or more dimensions. The deflection angle is defined as an angle between: an incident portion of the primary beam along a first optical axis of one or more upstream optical elements of the beam delivery system, wherein the upstream optical elements are arranged in a beam path of the primary beam upstream of the stationary deflection element, and a deflected portion of the primary beam along a second optical axis of one or more downstream optical elements of the beam delivery system, wherein the downstream optical elements are arranged in the beam path of the primary beam downstream of the stationary deflection element. Furthermore, the deflection angle is larger than 90°.

Thus, the ophthalmic laser system may provide an improved flexibility in the design and arrangement of individual parts and/or the whole ophthalmic laser system. In case the angle is provided with a deflection angle >90°, additional space may be created between the components of the beam ophthalmic laser system, in particular between the beam delivery system and the patient. The additional space may be created at a bottom side of the components of the ophthalmic laser system, which may include a bottom side of the beam delivery system facing the patient. In different embodiments the deflection angle may be also smaller than 90°, which may accordingly allow creating space at the opposite side, e.g. at an upper side of the components of the ophthalmic laser system, for instance an upper side of the beam delivery system facing away from the patient.

The beam delivery system may include an objective lens system comprising one or more lenses adapted for focusing the treatment laser beam within the eye. At least a portion of the objective lens system may be part of a movable portion of the beam delivery system. The upstream and/or downstream optical elements may be configured to focus the treatment laser beam within the eye. Further, the downstream optical elements may be adapted to let the laser beam exit in a desired manner from the beam delivery system toward the contact element of a patient interface.

The scanning system may be operatively coupled with a controller. The controller may be configured to determine a scanning path of the pulsed laser beam for scanning the laser focus on or within the cornea or within the natural lens of the patient's eye. The controller may be configured to determine the scanning path based on patient specific data. The controller may be configured to generate the scanning pattern so that the laser pulses are overlapping or non-overlapping. A lateral displacement of neighbouring laser pulses may be less than 30 micrometers, or less than 20 micrometers, or less than 10 micrometers. The lateral displacement may be greater than 0.5 micrometers or greater than 1 micrometer.

The primary beam may be created by a laser source, which may include an oscillator laser, which is configured to generate a train of low energy ultrashort pulses. A pulse energy of the low energy ultrashort pulses may be lower than 100 nJ or lower than 20 nJ, or lower than 10 nJ. The pulse energy may be greater than 1 pJ or greater than 100 pJ. The laser source may also include an amplifier, such as a regenerative amplifier or fibre amplifier for amplifying at least a portion of the low energy ultrashort pulses.

A base portion or base of the laser system may house a portion of the laser source. By way of example, the oscillator laser may be arranged in the base and the amplifier and/or a pre-compensator of the laser source, which is configured to reduce a group delay dispersion of the treatment laser beam may be arranged in the laser applicator. Alternatively, the base portion may house the entire laser source, such as the oscillator laser, the amplifier and the pre-compensator.

The base portion may include one or more housings. Each of the housings may house a portion of the laser source. By way of example, a first housing of the base may house a seed laser of the laser source and a second housing of the base may house an amplifier of the laser source, in particular a regenerative amplifier or a fibre amplifier.

The scanning system may provide three degrees of freedom for positioning the focus of the laser treatment beam at different locations within the eye or on the anterior surface of the cornea. One of the three degrees of freedom may be provided by an axial scanning system. The remaining two degrees of freedom of the scanning system may be provided by a beam deflection scanning system.

The axial scanning system may be configured for scanning the laser focus along an axis of the laser beam. The beam deflection scanning system may be configured for scanning the laser beam through deflection of the laser beam. The axial scanning system may be in the beam path of the laser beam between the laser source of the ophthalmic laser system and the beam deflection scanning system. The scanning system may be in the beam path of the treatment laser beam between the laser source and the beam delivery system.

The laser source may be an infrared laser source. The infrared laser source may be configured as a femtosecond laser source. A pulse energy of the laser pulses may be greater than 1 nanojoule, or greater than 10 nanojoule or greater than 50 nanojoule. The pulse energy may be less than 20 microjoule, or less than 15 microjoule or less than 10 microjoule. A pulse duration of the pulsed laser beam may be less than 800 femtoseconds, or less than 500 femtoseconds, or less than 300 femtoseconds, or less than 150 femtoseconds, or less than 100 femtoseconds. The pulse duration may be greater than 10 femtoseconds or greater than 50 femtoseconds. A repetition rate of the pulsed laser beam may be greater than 50 kHz or greater than 80 kHz. The repetition rate of the pulsed laser beam may be less than 10 MHz or less than 1 MHz. A centre wavelength of the pulsed laser beam, which is incident on the eye may be in a range of between 800 nanometers and 1400 nanometers, or between, 900 nanometers and 1400 nanometers, or between 1000 nanometers and 1100 nanometers, or between 1010 nanometers and 1050 nanometers.

The infrared laser source may be configured so that the laser pulses have a pulse energy so that the laser beam generates photodisruption within corneal tissue or within the natural lens of the patient's eye. The photodisruption may be caused by laser-induced optical breakdown. Alternatively, a pulse energy of the laser pulses may be below a threshold for generating laser-induced optical breakdown. By way of example, a plurality of pulses, which have a pulse energy below the threshold for generating laser-induced optical breakdown may be overlapped in a manner so as to generate tissue separation within the cornea.

The infrared laser source may include a pre-compensator for at least partially pre-compensating a change of the group delay dispersion (GDD) of the laser pulses, which is induced by components of the laser optical system, which are in the beam path of the laser beam downstream of the laser source. The pre-compensator may be configured to reduce the group delay dispersion. By way of example, the reduced group delay dispersion generated by the pre-compensator may have a lower positive or a more negative group delay dispersion.

In addition to a scanning movement of the laser focus along the axis of the laser beam, the axial scanning system may also cause a deflection of the laser beam so that the focus of the laser beam performs a lateral movement concurrently with the axial movement. The lateral movement of the laser focus may be smaller than the axial movement of the laser focus. A deflection of the treatment laser beam, which is performed using the beam deflection scanning system may adjust a lateral position of the laser focus relative to an optical axis of the beam delivery system.

The first optical axis may be defined by one or more upstream optical elements of the beam delivery system. In this respect, upstream may be understood as being arranged before the stationary deflection element in light of a treatment laser beam emittance direction from the laser source.

The second optical axis may be defined by one or more downstream optical elements of the beam delivery system. In this respect, downstream may be understood as being arranged after to the stationary deflection element in light of a treatment laser beam emittance direction from the laser source.

The beam delivery system may comprise one or more exit optical elements through which the treatment laser beam may exit from the beam delivery system to travel toward a patient interface. During treatment, the optical axis of a movable portion of the beam delivery system may be aligned or substantially aligned with the axis of the eye to be treated.

A lateral diameter of the focus of the treatment laser beam of the infrared laser source within the cornea or the lens may be smaller than 10 micrometers, or smaller than 6 micrometers. The diameter may be greater than 3 micrometers. The lateral diameter may be measured in a direction perpendicular to an optical axis of the laser optical system. The lateral diameter may be measured as an 80% encircled energy diameter.

Alternatively, the laser source may be configured as an UV laser source, e.g. as an excimer laser source. The laser system may be configured to generate a laser beam, which is focused on the anterior surface of the eye. A wavelength of the treatment laser beam generated by the excimer laser source may be greater than 150 nm or greater than 190 nm. The wavelength may be less than 400 nm or less than 200 nm. A pulse duration of the treatment laser beam generated by the excimer laser source may be shorter than 100 ns or shorter than 50 ns. The pulse duration may be greater than 1 ns or greater than 3 ns.

The laser may be focused within the eye by the scanning system, which may include one or more scanning paths. Examples for such scanning paths are but are not limited to: scanning paths within in the cornea, scanning paths for performing anterior capsulotomy (i.e. forming a flap or an opening in the lens capsule), scanning paths for posterior capsulotomy (i.e. for forming an opening in the posterior lens capsule) and/or scanning paths for lens fragmentation.

Examples for scanning paths within the cornea are scanning paths for forming an anterior surface lamella, (such as LASIK flaps), an intrastromal lamella (such as in SMILE and FLEx procedures) or a posterior surface lamella (such as in endothelial keratoplasty procedures). SMILE is an abbreviation for “small incision lenticule extraction”, FLEx is an abbreviation for “femtosecond lenticule extraction” and LASIK is an abbreviation for “laser-assisted in-situ keratomileusis”. Further examples are scanning paths for forming limbal relaxing incisions (LRI) and cataract incisions for providing access to the anterior chamber during cataract surgery.

The stationary deflection element may be understood as an element that may enable a deflection of the primary beam, which is irradiated to the stationary deflection element at a certain angle, toward the eye at a fixed output angle. “Stationary” in this context may be understood such that the stationary deflection element is essentially not moved or tiled during the laser treatment. Of course, the output angle may vary, e.g. during performing the laser treatment, depending on the input angle of the primary beam. Nevertheless, the stationary deflection element may of course also be moved to find a suitable initial position or orientation with respect to an eye to be treated.

According to a further embodiment, the deflection angle provides an increased distance or an increased free space between the patient and one or more of the plurality of components of the ophthalmic laser system compared to a 90° deflection angle.

Thus, the ophthalmic laser system may have an increased flexibility in the setup and arrangement with respect to the patient. It is noted in this respect that a “free space” not necessarily means that no elements are provided in said “free space”. Quite to the contrary, the distance or free space may be advantageously increased such that additional apparatus or other elements used for performing laser treatments could be provided in therein. The present invention is not delimited thereto, and the additional free space may also be used for any other suitable purpose, e.g. a provision, storage or supply of diagnostic or treatment equipment of any kind. Hence, the increased distance or space may be understood as an additional space, that otherwise would be occupied by parts of an ophthalmic laser system that operates at a 90° deflection angle. The distance and increased free space may be for instance determined from an outermost body part of the patient facing the beam delivery system. Or the increased distance and increased free space may be for instance determined from a plane extending parallel to the floor in a height of an outermost body part of the patient facing the beam delivery system.

According to a further embodiment, the beam delivery system comprises a patient interface portion comprising a patient interface for docking the eye to the ophthalmic laser system, wherein the increased distance or the increased free space is provided upstream of the patient interface portion.

Thus, the increased distance or free space may be obtained even when the patient is docked with the eye to the ophthalmic laser system. This may allow to achieve the increased distance or free space between the patient and the ophthalmic laser system also during performance of the laser treatments. During the laser treatment, the patient's eye may be docked to the portion of the beam delivery system e.g. via a contact element of a patient interface, wherein the contact element may contact an anterior surface of the cornea of the patient's eye and the treatment laser may traverse the contact element after having exited the beam delivery system. The docking may provide a reversible mechanical connection between the eye and the ophthalmic laser system.

According to a further embodiment, the optical elements comprise one or more lenses, and wherein the ophthalmic laser system further comprises a lens mount arranged at a lens mount portion of the beam delivery system upstream of the patient interface portion, wherein the lens mount is adapted to hold one or more of the upstream optical elements, wherein the increased distance or the increased free space is provided between the patient and the lens mount.

Thus, an increased distance or free space may be obtained between the lens mount and the patient. A lens may include any suitable optical element that may allow a respective beam manipulation. This may include, but is not limited to, lenses which may have one or more of at least partially concave, convex and/or planar portions. The lenses may comprise any suitable materials that allow at least a proper partial beam propagation therethrough. The lens mount may be adapted to hold the one or more optical elements, such as lenses and/or mirrors in place such that setting a defined beam propagation may be enabled. The lens mount may accordingly comprise means for holding at least a portion of the one or more optical elements lenses in place in a removable or non-removable manner. The arrangement of the lens mount and the respective optical elements disposed therein upstream of the patient interface portion may accordingly allow to direct a primary beam towards the stationary deflection element and the patient interface portion. Hence, an increased distance or the increased free space may be provided between the patient and the lens mount portion of the beam delivery system.

According to a further embodiment, the beam delivery system comprises a housing, wherein the housing is adapted to accommodate at least partially one or more of the plurality of components of the ophthalmic laser system, wherein the housing comprises an inclined portion upstream of the patient interface portion, wherein the inclined portion faces towards the patient, wherein the increased distance or the increased free space is provided between the patient and the inclined portion of the housing.

Thus, an increased distance or free space may be obtained between the housing and the patient. The housing may be adapted to at least partially or fully enclose or protect the one or more of the plurality of components of the ophthalmic laser system from environmental impacts such as dust and moisture. The inclined portion upstream of the patient interface portion may be understood as being inclined with respect to a patient or to a plane parallel to a main extension direction of the patient for instance parallel to the ground or a patient support, such as a table, upon which the patient is arranged. The inclination may at least partially, preferably essentially, lead to an increasing distance from the patient with increasing distance from the patient interface portion. In other words, the distance between the patient and the beam delivery system may increase with increasing distance from the patient interface portion where the patient's eye is docked to the patient interface during laser treatment.

According to a further embodiment, the inclined portion of the housing is located in contact or at a distance in a range of 0.5 mm to 20 mm to the lens mount; preferably in a range of 1.0 mm to 15 mm, and most preferably in a range of 1.75 mm to 10,9 mm and/or at least a portion of the inclined portion of the housing is formed by at least a portion of the lens mount.

Thus, the inclined portion of the housing may be arranged in close proximity to the lens mount and/or may be even formed by the lens mount. Accordingly, the inclined portion of the housing and/or the lens mount may be suitably designed to achieve an increased distance or free space between the housing and/or the lens mount. In some examples, the whole inclined portion of the housing may be formed by least a portion or the whole lens mount.

According to a further embodiment, the deflection angle is larger than 95°, preferably larger than 100°, more preferably larger than 105°. Thus, an even further increased distance or free space may be achieved compared to a 90° deflection angle. Of course, also even larger angles may be achieved, if needed to increase the additional space or distance even more.

According to a further embodiment, the deflection angle is smaller than 180°, preferably smaller than 175°, more preferably smaller than 170°.

Thus, a compact design of the ophthalmic laser system and in particular of the beam delivery system may be obtained while an increased free space or distance can be achieved.

According to a further embodiment, the primary beam comprises a single beam or a plurality of beams.

Thus, one or more of the beams of the primary beam may be accordingly focused on the eye to be treated. The respective optical elements may be accordingly adapted to suitably manipulate the respective beam paths dependent on the properties of the one or more beams, for instance a wavelength of the respective beam. Or, the respective optical elements may be adapted to suitably manipulate the beam paths for the different purposes, such as directing the laser treatment beam one the one hand to the location to be treated, and, on the other hand, directing another beam, such as an OCT beam and/or an optical imaging beam to a location to be imaged, which may be the same location or a different location as the location to be treated.

According to a further embodiment, the primary beam is deflected so that downstream of the stationary deflection element, the primary beam travels along the second optical axis, which is substantially parallel to an axis of the eye.

Thus, the primary beam may be suitably guided toward one or more areas of interest of the eye to be treated. The term “axis of the eye” as used herein may refer to the visual axis of the eye or to the optical axis of the eye. The term “optical axis of the eye” as used herein may refer to an axis, which is oriented normal to the anterior surface of the cornea, and which has the smallest distance from the centre of curvature of all remaining refractive surfaces of the eye. The term “visual axis of the eye” (also referred to as “line of vision”) as used herein may refer to the straight line extending from the object seen, through the centre of the pupil, to the macula lutea of the retina. Substantially parallel may also include a primary beam that is parallel the axis of the eye or a primary beam that travels along the axis of the eye.

According to a further embodiment, an interior housing surface of at least a portion of the inclined portion of the housing faces toward at least a portion of the lens mount.

A facing of the inclined portion of the housing towards the portion of the lens mount may include an immediate or direct facing between respective surfaces of the housing and the portion of the lens mount with no elements in between. However, the “facing” of the inclined portion of the housing towards the portion of the lens mount may also include an indirect facing between respective surfaces of the housing and the portion of the lens mount with elements arranged in between. In some examples, the whole inclined portion of the housing may face toward a portion of the lens mount or the whole lens mount.

According to a further embodiment, at least a portion of the interior housing surface follows an exterior lens mount surface portion of the lens mount.

Hence, an interior housing surface may be arranged in the vicinity of the lens mount thus allowing for a particular compact arrangement. In some examples, the whole interior housing surface may follow an exterior lens mount surface portion of the lens mount.

According to a further embodiment, at each location of the exterior lens mount surface portion, the inclined portion of the housing has a thickness which is in a range of 0.5 mm to 7 mm.

Hence, a space saving housing may be provided, which simultaneously may enable sufficient mechanical stability for protecting the housed parts.

According to a further embodiment, at least a portion of an exterior housing surface of at least a portion of the inclined portion of the housing faces a portion of the patient, and wherein the exterior housing surface follows at least partially a shape of the portion of the patient.

Thus, the housing may be accordingly suitably designed to meet a shape of at least portions of a patient. For instance, the exterior housing surface may follow a shape of a head or a chest portion of a patient. Thus, the ophthalmic laser system may be operated in a particular space-saving manner. In some examples, the whole exterior housing surface of a portion or the whole of the inclined portion may face a portion of the patient. In some examples, the exterior housing surface may completely follow a shape of the portion of the patient.

According to a further embodiment, an additional free space is created by the inclined portion for allowing a user to perform surgical operations.

Thus, no further parts may be arranged in the additional free space, but the free space may be employed by for instance by a surgeon performing surgical operations. Surgical operations may include any medical actions performed for instance by medical professionals, e.g. movements or gestures a surgeon has to perform during operation.

According to a further embodiment, the exterior housing surface is formed such that a clearance between the patient and the housing is maximized.

Thus, the largest clearance possible may be obtained for the ophthalmic laser system employed.

According to a further embodiment, the scanning system is arranged in the beam path of the primary beam upstream of the upstream optical elements.

Thus, the primary beam may be suitably guided and manipulated by the scanning system to allow irradiating it at one or more desired locations toward the upstream optical elements arranged downstream of the scanning system.

According to a further embodiment, the scanning system comprises: an axial scanning system for three-dimensional positioning of a focus of the primary beam in a treatment volume of the eye of the patient.

The axial scanning system may be configured to axially scan the focus of the laser beam through varying an angle of divergence or convergence of the laser beam. The divergence or convergence may be measured at a location along the axis of the laser beam, where the laser beam exits from the axial scanning system. The terms “divergence” or “convergence” as used herein may be defined to mean an angular measure of the increase or decrease in beam diameter with distance.

The axial scanning system may include one or more displaceable lenses, which are arranged in the beam path of the treatment laser beam. The axial scanning system may be configured so that the one or more displaceable lenses are controllably displaceable in a direction parallel or substantially parallel to an optical axis of the one or more displaceable lenses. The ophthalmic laser system may include a controller, which is in signal communication with an actuator of the axial scanning system. The actuator may be configured to displace the one or more displaceable lenses based on signals received from the controller.

The axial scanning system may include a first optical system, which has a negative optical power; and a second optical system, which has a positive optical power. The second optical system may be in the beam path of the laser beam between the first optical system and the deflection scanning system. The axial scanning system may further be configured so that a distance between the first optical system and the second optical system is controllably variable.

According to a further embodiment, a diameter of three or four upstream optical elements arranged closest to the stationary deflection element along the first optical axis continuously decreases toward the stationary deflection element.

According to a further embodiment, an outer shape of the lens mount and/or the housing substantially tapers towards the stationary deflection element.

Thus, a cone-like appearance may be achieved for the outer shape of the lens mount and/or the housing. Accordingly, larger optical elements may be arranged in a greater distance to the stationary deflection element and small optical elements may be arranged in closer proximity to the stationary deflection element.

According to a further embodiment, the beam delivery system is part of a displaceable laser applicator, wherein the ophthalmic laser system comprises a support arm for displaceably supporting the laser applicator.

The support arm may be configured so that a distance measured along a horizontal between the laser applicator and a location where the support arm is connected to the base may be adjustable. A maximum horizontal distance between the base and the applicator head may be greater than 50 centimeters or greater than 10 centimeters. The distance may be smaller than 5 meters or smaller than 3 meters.

According to a further embodiment, the support arm or at least an arm segment of the support arm is configured to be rotatable about a horizontal or substantially horizontal rotation axis and/or a vertical or substantially vertical rotation axis.

The support arm may include one or more arm segments. The support arm may include a plurality of arm segments, which are connected in series. Neighbouring arm segments of the support arm may be connected via one or more joints. An arm segment may be defined as a member of the support arm, which provides a non-articulated connection between a first end of the arm segment and a second end of the arm segment. The arm segment may provide a rigid or telescoping coupling between the first end of the arm segment and the second end of the arm segment. At one or at both ends of the arm segment, the arm segment may be attached or integrally connected to a joint. The term “integrally connected” may indicate that a first element/feature extends or transitions in a continuous manner from a second element/feature and not as two separate and distinguishable elements.

The support arm may further be configured so that the laser applicator is movable in a circular or substantially circular arc path of travel. The arc may be in a horizontal or substantially horizontal plane. A radius of the arc may be greater than 10 centimeters or greater than 50 centimeters. The radius may be smaller than 5 meters or smaller than 3 meters. The radius may be defined by a longitudinal extent of an arm segment of the support arm, wherein the support arm is configured so that the arm segment can be rotated about a horizontal rotation axis and/or about a vertical rotation axis. Alternatively, the support arm may be configured so that the arm segment can be rotated about a substantially horizontal rotation axis and/or about a substantially vertical rotation axis. The arm segment may be part of a multi bar linkage, in particular part of a four bar or parallel linkage. Additionally or alternatively, the arm segment may be balanced for providing at least partial gravity counterbalancing for the laser applicator.

According to a further embodiment, the support arm is movably hinged at a base portion of the ophthalmic laser system so that the laser applicator is positionable relative to the base portion in one or more, in particular three, dimensions.

The support arm may be connected to the laser applicator at a first end. The second end of the support arm may be connected to the base and/or may be connectable to a further component at the second end of the support arm. The further component may be external to the ophthalmic laser system. By way of example, the further component may be a second base or may be a wall, a ceiling or a floor of a building.

The base portion of the ophthalmic laser system may be configured as a moveable base, which is moveable across the floor surface. The moveable base may include a plurality wheels, such as four wheels for moving the base across the floor surface. The moveable base may be configured so that the entire laser surgical system can be moved by one person across the floor. The support arm and the laser applicator may only be supported by the base portion.

According to a further embodiment, the laser applicator is positionable relative to the base portion while maintaining an essentially vertical orientation of the laser applicator.

Consequently, a desired fixed orientation of the laser applicator may be achieved, which may facilitate the relative arrangement of the laser applicator with respect to the eye of a patient.

According to a further embodiment, a positioning of the laser applicator relative to the base portion includes a rotation of the laser applicator relative to the base portion in one or more, in particular three, dimensions.

Thus, an independent rotation of the laser applicator may be achieved.

According to a further embodiment, the support arm comprises a first arm segment and a second arm segment which are connected to each other in series via an intermediate joint; wherein the first arm segment is rotatable about a vertical or substantially vertical axis and the second arm segment is rotatable about a horizontal or substantially horizontal axis.

According to a further embodiment, the intermediate joint is configured to allow the second arm segment to rotate about the horizontal or substantially horizontal axis and about a vertical or substantially vertical axis.

Thus, a flexible spatial arrangement and movement of the support arm and the respective segments may be achieved.

According to a further embodiment, the ophthalmic laser system comprises a controller operatively coupled with the beam delivery system and configured to control the scanning system to position a focus of the primary beam at different locations within the eye or on an anterior surface of the cornea of the eye.

The controller may include a data processing system. The data processing system may include a computer system having one or more processors and a memory for storing instructions processable by the processor. The one or more processors may execute an operating system. The data processing system may further include a user interface configured to allow a user to receive data from the data processing system and/or to provide data to the data processing system. The user interface may include a graphical user interface. The ophthalmic laser system may include a pointing device, such as a computer mouse. The graphical user interface may be configured for receiving user input via the pointing device.

According to a further embodiment, the ophthalmic laser system comprises an optical measuring system for acquiring measurement data of at least a portion of the eye, wherein at least one beam of the optical measuring system is combined with the primary beam by the stationary deflection element.

Thus, multiple beams of different systems may be employed during operation of the ophthalmic laser system and may be directed to the eye to be treated. Thus, complex surgical operations may be performed and the treatment with the primary beam may be enhanced. An optical measuring system may include any system that may allow acquiring optical data of at least a portion of the eye before, during or after the laser treatment. In some examples, measurement data of the whole eye may be acquired by the optical measuring system.

According to a further embodiment, the ophthalmic laser system comprises an optical measuring system for acquiring measurement data of at least a portion of the eye, wherein at least one beam of the optical measuring system is combined with the primary beam upstream of the stationary deflection element.

Thus, a compact arrangement of the ophthalmic laser system could be obtained, since no further means are necessary to combine the primary beam with the at least one beam of the optical measuring system. A beam combination may be understood as adapting the beam paths of the primary beam and the at least one beam of the optical measuring system to travel at least partially parallel to each other or that the primary beam and the at least one beam of the optical measuring system may travel along the same beam path. In some examples, all beams of the optical measuring systems may be combined with the primary beam upstream of the stationary deflection element.

According to a further embodiment, the optical measuring system comprises an optical coherence tomography system configured for acquiring a cross-sectional image of at least a portion of the eye.

A centre wavelength of the optical coherence tomography—OCT—system may be within a range of between 750 and 1400 nanometers. The optical coherence tomography system may be configured to acquire a cross-sectional image of the cornea and/or at least a portion of the natural lens of the eye. In some examples, a cross-sectional image of the whole eye may be acquired by the optical coherence tomography system.

According to a further embodiment, the OCT system includes an OCT beam scanner for scanning an OCT beam along one or more dimensions across the eye.

This may include a scanning along one or more axes in a plane, e.g. an x-y-plane, as well as a scanning along a direction perpendicular thereto, e.g. in a z-direction.

According to a further embodiment, the OCT beam scanner is separate from the scanning system of the primary beam. According to a further embodiment, the OCT beam scanner is integrated in the scanning system of the primary beam.

Thus, the OCT beam scanner may be flexibly provided in a desired external or integrated manner.

According to a further embodiment, the optical measuring system comprises an optical imaging system for acquiring a frontal image of at least a portion of the eye.

The optical imaging system may acquire a frontal image of the eye to be treated, in particular an image of a region surrounding the optical axis of the beam delivery system at a location within the anterior segment of the eye. By way of example, the imaged region may include the pupil and/or at least a portion of the iris of the eye to be treated. In some examples, a frontal image of the whole eye may be acquired by the optical imaging system. The imaging beam path may extend through one or more optical elements of the patient interface, in particular through a contact element of the patient interface, which contacts an anterior surface of the cornea.

Furthermore, an additional camera may be provided, wherein the additional camera may be adapted to observe a patient's head and/or a second eye of the patient to enable a rotational compensation.

The controller of the ophthalmic laser system may include an image processing algorithm for determining, whether at least a portion of the frontal image is in focus. Additionally, or alternatively, the image processing algorithm may be configured to determine one or more parameters, which depend on, or which are indicative of, a level of focus of at least a portion of the image. The image processing algorithm may include a segmentation algorithm for segmenting the frontal image. The image processing algorithm may determine one or more parameters, which depend on, or which are indicative of, a level of focus of one or more of the segmented image regions.

The optical imaging system may acquire a frontal image of at least a portion of the eye to be treated, at least during a state in which the eye is docked or substantially docked to the ophthalmic laser system. The optical imaging system may include an image sensor and an imaging optical system for imaging the portion of the eye onto the image sensor. The image sensor may include a two-dimensional ordered or unordered array of pixels. The image sensor may be sensitive to one or more wavelengths within a range of between 380 nanometers and 950 nanometers or within a range of between 380 nanometers and 1400 nanometers. The imaging system may include an imaging optical system for imaging a tissue portion which is arranged in an object plane of the imaging optical system onto the image sensor. At least a portion of the imaging optical system may be provided by a portion of the beam delivery system for focusing the treatment laser beam into the eye of the patient.

A distance of an object plane of the imaging system from the laser applicator may be configured, or may be adjustable so that, the distance substantially corresponds to a distance of the laser applicator from the cornea of the patient's eye during the laser treatment.

According to a further embodiment, the optical measuring system further comprises a camera.

Thus, a particular image information may be additionally gathered. The camera may any suitable camera, e.g. a digital camera. The camera might be connected to imaging processing means, which may allow to process acquired image data and present the image data to a user of the ophthalmic laser system. Such image data may be for instance enhanced by the image processing means executing respective image processing software which may allow to manipulate the acquired image data as desired.

According to a further embodiment, the optical measuring system further comprises a confocal microscope.

The optical coherence system and/or confocal microscope may be configured to acquire one or more cross-sectional images of the cornea and/or of at least a portion of the natural lens of the eye. The image planes of the cross-sectional images may be non-parallel to each other. The ophthalmic laser system may be configured to allow the user and/or the controller to use the images of the non-parallel image planes to align the axis of the eye to be treated with the optical axis of the movable portion of the beam delivery system. In one example, the treatment laser beam may be employed by the confocal microscope.

According to a further embodiment, the stationary deflection element is part of a first beam combiner configured to combine a beam path of the primary beam and at least one secondary beam path of at least one secondary beam.

Thus, a particular compact arrangement may be obtained, since no further means are necessary to combine the beam path of the primary beam and the at least one secondary beam path of the at least one secondary beam. In some examples, a beam path of the primary beam may be combined with all beam paths of all secondary beams. In other words, the beam paths of one or more primary beams and one or more secondary beams could be combined by the stationary deflection element. The beam combiner may be arranged in the beam path of the laser beam between the scanning system and the eye. The beam combiner may be configured for combining the beam path of a primary beam, for instance a treatment laser beam, with the beam path of a secondary beam, for instance an imaging beam. The stationary deflection element may be arranged in a fixed manner with respect to one or more further elements of the first beam combiner.

According to a further embodiment, one or more, preferably all, elements of the first beam combiner are made of an optical material with refractive index above 1.7, more preferably in a range of 1.77 to 1.84.

According to a further embodiment, the OCT beam scanner is arranged upstream the first beam combiner.

Hence, a beam path of the OCT system may be combined with a beam path of a primary beam, e.g. with the beam path of the treatment laser beam. The beam combiner may include a reflection prism as described herein, and the OCT beam path may be accordingly combined with the beam path of the treatment laser beam.

According to a further embodiment, the secondary beam path comprises one or more beam paths of beams of the optical measuring system.

A second beam combiner may be provided upstream of the first beam combiner. The second beam combiner may include or consist of a dichroic mirror, which may be adapted to combine a beam path of a first secondary beam, which may be for instance a beam path of the OCT system with a beam path of a second secondary beam, which may be for instance a beam path of a beam of an optical measuring system. The secondary beams accordingly combined in a combined beam path and may be further combined with a beam path of a primary beam by the first beam combiner.

According to a further embodiment, the stationary deflection element comprises a reflection prism for deflecting the primary beam.

Thus, the beam path of the primary beam may be suitably directed by the stationary deflection element. The deflection may be achieved by deflecting the primary beam at a certain angle from an incidence direction to an output direction.

According to a further embodiment, the reflection prism is further configured for deflecting the secondary beam via one or more internal reflections from a second incidence direction towards an output direction.

Thus, also the beam path of the secondary beam may be suitably directed by the stationary deflection element. Further, since the secondary beam may be deflected by internal reflections, no further means, such as additional reflective surfaces may be necessary to deflect the secondary beam.

According to a further embodiment, the stationary deflection element is part of the first beam combiner. According to a further embodiment, the stationary deflection element is formed by the reflection prism, wherein the reflection prism is a beam combining prism configured to combine the primary beam and the secondary beam to a common beam path along the output direction.

Thus, a reduced complexity and increased robustness of the ophthalmic laser system may be achieved. In particular, no further additional elements may be necessary to combine the primary beam and the secondary be to a common beam path along the output direction, as this is achieved by the beam combining prism. The beam combining prism may be accordingly arranged in a fixed manner with respect to one or more further elements of the first beam combiner.

According to a further embodiment, the reflection prism is adapted to reflect the secondary beam internally twice from the second incidence direction towards the output direction.

Thus, the travel direction of the beam may be advantageously directed towards the output direction via two internal reflections.

According to a further embodiment, the ophthalmic laser system further comprises an illumination device configured for illuminating at least a portion of the eye.

Thus, portions of interest of the eye to be treated may be suitably illuminated. This may enhance the treatment abilities with the ophthalmic laser system. In some examples, the illumination device may be configured to illuminate the whole eye.

According to a further embodiment, the illumination device includes one or more light sources adapted to emit one or more secondary beams towards a reflection prism, wherein the one or more secondary beams comprise illumination light beams.

The illumination device may be configured to illuminate at least a portion of the eye. Further, a position and/or an orientation of a light emitting surface of the light source may be indicative of or correlated with an orientation of a movable portion of the beam delivery system. Additionally, or alternatively, a direction of light emitted by the light source may be indicative of or correlated with the orientation of the movable portion of the beam delivery system. The direction of light may have a fixed orientation relative to the movable portion of the beam delivery system. The orientation may be measured in a plane perpendicular to the axis of the eye to be treated.

According to a further embodiment, the illumination device comprises at least one LED light source. According to a further embodiment, the LED light source is formed as a ring extending in a plane essentially parallel to a surface of the reflection prism upon which the illumination light beams are irradiated.

Thus, a particular efficient and space-saving arrangement of the illumination device could be achieved. In some examples, the illumination device may be adapted to create Purkinje images that may be employed during the operation of the laser treatment. In some examples, also more than one LED light source may be provided. In some examples, the controller may be enabled to individually switch individual LEDs or groups of LEDs and may allow to record synchronized images.

According to a further embodiment, the reflection prism is adapted to deflect a beam path of the secondary beam from the second incidence direction towards the output direction in a range of about 45° to 85°, preferably of about 50°. According to a further embodiment, the reflection prism is a Bauernfeind prism.

Thus, the beam paths may be accordingly guided such that an image of the eye may be advantageously neither flipped or rotated when being deflected.

According to a further embodiment, the reflection prism has a first prism side facing the primary beam coming from a first incidence direction, a second prism side facing one or more secondary beams coming from the second incidence direction, and a third prism side facing one or more reflected beams of the one or more secondary beams reflected or back scattered by the eye along a reflected or back scattered direction.

Each of the first prism side, the second prism side and third prism side may contact the respective other two prism sides, preferably along a straight edge. In a cross-sectional view of the reflection prism, the first prism side, the second prism side in the third prism side may essentially form a triangle.

According to a further embodiment, one or more of the first prism side, the second prism side and the third prism side include reflective surfaces comprising at least partial optical coatings for reflecting the primary beam and/or the secondary beam.

Thus, an enhanced reflection of the primary beam and/or the secondary beam may be achieved. The coatings may be accordingly adapted to the properties and nature of the beams employed and the one or more prism materials. The same coating may be provided for the primary beam in the secondary beam. Or different coatings may be provided, which may be adapted to the respective one of the first and/or secondary beam. As a mere example, a first coating may be provided to properly essentially reflect the primary beam while being essentially transparent to the secondary beam and a second coating may be provided to properly essentially reflect the secondary beam while being essentially transparent to the primary beam. The coatings may be accordingly adapted to reflect the respective beams totally or only partially. In some examples, one or more of the first prism side, the second prism side and the third prism side may be fully covered by respective optical coatings for reflecting the primary beam and/or the secondary beam.

According to a further embodiment, one or more of the first prism side, the second prism side and the third prism side are additionally or alternatively adapted to for enabling a total internal reflection the primary beam and/or the secondary beam.

Thus, no additional reflective surfaces may be necessary to reflect the primary beam and/or the secondary beam. The respective surfaces may be accordingly adapted to allow suitable incident and output directions for the respective beams to achieve total internal reflection.

According to a further embodiment, one or more of the optical coatings include dichroic, notch and/or partial coatings.

Thus, the optical coating may be suitably adapted to the needs of the optical system. For instance, the optical coating may be adapted to be transparent to the primary beam and to reflect the secondary beam or vice versa. Or the optical coatings may be adapted to the individual beams included in the primary beam or the secondary beam. Furthermore, partial coatings may only cover a portion of the respective surface. Notch coatings may transmit certain wavelengths and simultaneously block or reflect others.

According to a further embodiment, the first prism side and the second prism side contact each other at a functional prism angle, wherein the functional prism angle is in the range of 22.5° to 42.5°, preferably of about 25°.

According to a further embodiment, the reflection prism is adapted to guide the secondary beam along a combined beam path, wherein the secondary beam is deflected under a secondary beam deflection angle, wherein the secondary beam deflection angle is defined between the second incidence direction and the output direction, wherein the secondary beam deflection angle is in a range of 45° to 85°, preferably of about 50°.

Thus, the beam path of the secondary beam may be suitably folded or redirected from a first direction to a second direction via the reflection prism. Accordingly, devices emitting and/or receiving the secondary beam, for instance an optical measuring system and/or an OCT system may be provided flexibly in different arrangements, in particular in an inclined manner with respect to the output direction. This may allow to provide the ophthalmic laser system in a particular space-saving manner. Furthermore, the devices emitting or receiving the secondary beam may be provided essentially facing a different side of the reflection prism compared to the devices emitting or receiving the primary beam.

According to a further embodiment, the reflection prism is adapted to guide the primary beam along a primary beam path, wherein the primary beam is deflected under a primary beam deflection angle, wherein the primary beam deflection angle is defined between the first incidence direction and the output direction.

Thus, the beam path of the primary beam may be suitably folded or redirected from a first direction to a second direction via the reflection prism. Accordingly, devices emitting and/or receiving the primary beam, for instance the treatment laser beam source and/or an OCT system may be flexibly provided in different arrangements, in particular in an inclined manner with respect to the output direction. This may allow to provide the ophthalmic laser system in a particular space-saving manner. Furthermore, the devices emitting or receiving the primary beam may be provided essentially facing a different side of the reflection prism compared to the devices for emitting or receiving the secondary beam.

According to a further embodiment, the functional prism angle, the secondary beam deflection angle and the primary beam deflection angle follow the relationships: β=2*α and γ=2*β.

According to a further embodiment, the second incidence direction is essentially perpendicular to the second prism side.

Thus, potential reflections at the second prism side may be mitigated and a lossless entry of the secondary beam entering the reflection prism via the second prism side may be achieved. Furthermore, a coupling of the secondary beam into the reflection prism may be improved. Furthermore, refractions of the secondary beam when entering the prism may be avoided.

According to a further embodiment, the reflected or back scattered direction is essentially perpendicular to the third prism side or wherein the reflected or back scattered direction is inclined at an angle of up to 45° with respect to a direction perpendicular to the third prism side.

Thus, potential reflections at the third prism side may be mitigated and a lossless entry of the reflected or backscattered primary or secondary beam entering the reflection prism via the third prism side may be achieved. Further, a coupling of the reflected or backscattered manner primary or secondary beam into the reflection prism may be improved. Furthermore, refractions of the primary or secondary beams when entering the prism may be avoided.

According to a further embodiment, the reflection prism is configured to couple the primary beam into the reflection prism at the first prism side such that the primary beam travels through the reflection prism towards the second prism side and is then internally reflected at the second prism side towards the third prism side, and wherein the primary beam is then coupled out of the reflection prism at the third prism side towards the eye.

Thus, the reflection prism may provide a particular compact and space-saving beam deflection system by redirecting the primary beam path without further elements necessary. In particular, one or more, preferably each, of the beams of the primary beam may be accordingly coupled into the reflection prism the first prism side and accordingly coupled out of the reflection prism the third prism side towards the eye.

According to a further embodiment, the primary beam is coupled out of the reflection prism essentially perpendicular to the third prism side along the output direction.

Thus, refractions of the one or more beams of the primary beam may be avoided when leaving the third prism side.

According to a further embodiment, the first prism side comprises a dichroic mirroring surface covering at least partially the first prism side, wherein the dichroic mirroring surface is at least partially transparent to the primary beam and at least partially reflective to the secondary beam having one or more different optical properties than the primary beam.

Thus, the primary beam may be guided at least partially through the first prism side and at the same time the secondary beam may be at least partially reflected or backscattered by the first prism side. Hence, a secondary beam may be reflected at an inner surface of the first prism side such that the secondary beam remains in the reflection prism and a primary beam may be allowed to enter the reflection prism via an opposite surface of the first prism side. Optical properties of the respective primary and secondary beams may include for instance a certain wavelength or wavelength ranges which may be accordingly different for the primary beam and the secondary beam. The primary beam may be accordingly an incoming beam that is directed onto the prism from the outside. The secondary beam may be accordingly a traversing beam that traverses the prism. In some examples, the first prism side may be fully covered by the dichroic mirroring surface. Further, in some examples, the dichroic mirroring surface may be fully transparent to the primary beam and/or fully reflective to the secondary beam.

According to a further embodiment, the reflection prism is configured to couple the secondary beam into the reflection prism at the second prism side such that the secondary beam travels through the reflection prism towards the first prism side and is reflected at the dichroic mirroring surface of the first prism side towards the second prism side, wherein the secondary beam then is internally reflected at the second prism side towards the third prism side, and wherein the secondary beam is coupled out of the reflection prism at the third prism side towards the eye.

Accordingly, a suitable beam path folding, or deflection may be obtained for the secondary beam from an incidence direction towards the second prism side to a different outcoupling direction from the third prism side towards the eye to be treated. The secondary beam may be accordingly outcoupled in the same direction as a primary beam, even though the secondary beam in the primary beam were emitted toward the prism at different prism sides.

According to a further embodiment, the outcoupled secondary beam is then at least partially reflected or back scattered by one or more features of the eye along a reflected or back scattered direction being essentially opposite to the output direction, wherein the reflection prism is configured to couple the reflected or back scattered secondary beam into the reflection prism at the third prism side such that the reflected or back scattered secondary beam travels through the reflection prism towards the second prism side and is then internally reflected at the second prism side towards the first prism side, wherein the reflected or back scattered secondary beam is then reflected at the dichroic mirroring surface of the first prism side towards the second prism side, and wherein the reflected or back scattered secondary beam is then coupled out of the reflection prism at the second prism side towards one or more detectors adapted to detect the reflected or back scattered secondary beam.

Thus, the reflection prism may not only allow to direct the secondary beam toward the eye but may likewise allow to guide reflected or backscattered radiation of the secondary beam back to respective detectors. The beam path for the reflected backscattered radiation of the secondary beam may be accordingly the same or similar as the beam path the secondary beam travelled in the initial incidence direction, however, in a reversed direction. Also, different beam paths at the reflection prism for the reflected or backscattered secondary beam may be provided. In some examples, the outcoupled secondary beam may be fully reflected backscattered by one or more features of the eye.

According to a further embodiment, the reflected or back scattered secondary beam is coupled out of the reflection prism essentially perpendicular to the second prism side.

Thus, refractions of the reflected or backscattered one or more beams of the secondary beam may be avoided when leaving the prism at the second prism side.

According to a further embodiment, the primary beam is coupled into the reflection prism at the first prism side at an angle in a range of 80° to 100°, preferably 85° to 95°, more preferably 82° to 92°.

According to a further embodiment, the primary beam is internally reflected at the second prism side at an angle in a range of larger than 90° to 170°, preferably of about 100°.

According to a further embodiment, the secondary beam and/or the reflected or back scattered secondary beam is reflected at the dichroic mirroring surface of the first prism side at an angle in a range of 45° to 85°, preferably of about 50°.

According to a further embodiment, the secondary beam and/or the reflected or back scattered secondary beam is internally reflected at the second prism side at an angle in a range of larger than 90° to 170°, preferably of about 100°.

Thus, suitable reflection angles may be provided to obtain a desired beam path folding of the primary beam and/or the secondary beam by the reflection prism.

According to a further embodiment, the first beam combiner comprises a dichroic mirror arranged in a beam path of the secondary beam upstream of the second prism side, wherein the dichroic mirror is at least partially transparent to a first secondary beam and at least partially reflective to a second secondary beam having one or more different optical properties than the first secondary beam, wherein the dichroic mirror is adapted to combine the first secondary beam and the second secondary beam along the second incidence direction.

Thus, one or more beams of the secondary beam may be guided at least partially through the dichroic mirror and at the same time other ones of the one or more beams of the secondary beam may be at least partially reflected by the dichroic mirror. Hence, the beam paths of the one or more beams of the secondary beam may be divided by dichroic mirror dependent on their optical properties. Optical properties of one or more beams of the secondary beam may include for instance a certain wavelength or wavelength ranges which may be accordingly different for other ones of the one or more of the secondary beam. In some examples, the dichroic mirror may be fully transparent to the first secondary beam. Furthermore, in some examples, the dichroic mirror may be fully reflective to the secondary beam.

According to a further embodiment, the downstream optical elements comprise a truncated cone-shaped optical element arranged between the stationary deflection element and the eye tapering along the second optical axis towards the eye.

Thus, the primary beam and/or the secondary beam may be guided along the extension direction, for instance parallel to or along a rotation axis of the truncated cone-shaped optical element from the stationary deflection element towards the eye. Thus, a defined beam path along the truncated cone-shaped optical element having defined optical properties may be provided, which may facilitate the guidance of the primary beam and/or the secondary beam toward the eye to be treated.

According to a further embodiment, the truncated cone-shaped optical element is part of the first beam combiner.

Thus, a particular compact and robust beam combiner could be provided, which may allow a distinct guidance of the primary beam and/or the secondary beam towards the eye to be treated. The truncated cone-shaped optical element may be arranged in a fixed manner with respect to one or more further elements of the first beam combiner.

According to a further embodiment, the truncated cone-shaped optical element comprises an essentially flat first cone end surface facing the third prism side and an oppositely arranged second cone end surface facing the eye.

Thus, the coupling of the primary beam and/or the secondary beam from the reflection prism to the truncated cone-shaped optical element and vice versa via the interface at the essentially flat first cone end surface and the third prism side may be provided. Likewise, an outcoupling of the primary beam and/or the secondary beam from the second cone end surface toward the eye may be enabled. The essentially flat first cone end surface may not be construed being fully flat, but may also be provided with a minor curvature to provide optimized optical properties for in-and outcoupling the primary beam and/or the secondary beam from and into the truncated cone-shaped optical element, respectively. Furthermore, also optimized treatment ranges and precisions in the positioning of the primary beam and/or the secondary beam in the eye to be treated may be considered by the shape of the essentially flat first cone end surface and the second cone end surface. The second cone end surface may be also essentially flat or may be curved in some embodiments.

According to a further embodiment, the essentially flat first cone end surface contacts at least partially the third prism side.

Thus, a predefined interface between the reflection prism and the truncated cone-shaped optical element may be obtained by a contact between the essentially flat first cone end surface and the third prism side. A contact may be either a direct contact between the essentially flat first cone end surface and the third prism side or may be also an indirect contact with further elements arranged in between the first cone end surface and the third prism side. In some examples, the essentially flat end surface may fully contact the third prism side.

According to a further embodiment, the patient interface is arranged between the second cone end surface and the eye.

Thus, the primary beam and/or the secondary beam coupled out of the second cone end surface of the truncated cone-shaped optical element may be guided to the eye via the patient interface.

According to a further embodiment, the truncated cone-shaped optical element is adapted to provide the primary beam to the eye at a numerical aperture of 0.1 to 0.4, preferably of 0.18 to 0.25, more preferably of about 0.2.

According to a further embodiment, the ophthalmic laser system further comprises at least one upstream optical coupling element arranged between the stationary deflection element and the upstream optical elements along the first optical axis.

Thus, the primary beam may be guided through the upstream optical coupling element and subsequently coupled into the stationary deflection element. In some examples, also more than one upstream optical coupling element may be provided. The optical coupling element may comprise suitable optical parameters with respect to the primary beam, such as a distinct transparency for the selected wavelengths of the one or more beams of the primary beam.

According to a further embodiment, the upstream optical coupling element is part of the first beam combiner.

Thus, a particular compact and robust first beam combiner may be provided since no additional external upstream optical coupling element is needed. The upstream optical coupling element may be accordingly arranged in a fixed manner with respect to one or more further elements of the first beam combiner.

According to a further embodiment, the upstream optical coupling element is an input prism lens.

Thus, the upstream optical coupling element may be suitably shaped and may comprise defined optical properties that may allow to guide the primary beam in a desired manner towards the reflection prism. The input prism lens may be in particular suitable for obtaining an optimized coupling of the primary beam into the reflection prism.

According to a further embodiment, the upstream optical coupling element comprises an essentially flat first upstream optical coupling element surface facing the first prism side and an oppositely essentially convexly curved second upstream optical coupling element surface facing the upstream optical elements.

Accordingly, the primary beam may be suitably guided from the outside to the essentially convexly curved second upstream optical coupling element surface through the upstream optical coupling element towards the first prism side. Further, the primary beam may then be coupled into the reflection prism at the interface between the first prism side and the essentially flat first upstream optical coupling element surface. The essentially flat first upstream optical coupling element surface may not be construed being fully flat, but it may also be provided with a minor curvature to provide optimized optical properties for in- and outcoupling the primary beam out of and into the reflection prism, respectively. Likewise, the essentially convexly curved second upstream optical coupling element surface may not be construed being only convexly curved, but it may also be provided at least partially with flat and/or concave portions to provide optimized optical properties for in- and outcoupling the primary beam out of and into the reflection prism, respectively.

According to a further embodiment, the essentially flat first upstream optical coupling element surface at least partially contacts the first prism side.

Thus, a particular optically well-defined interface between the essentially flat first upstream optical coupling element surface and the first prism side may be provided. Accordingly, a coupling of the primary beam into the reflection prism may be facilitated.

According to a further embodiment, the laser system is a stationary system or a mobile system.

Thus, the invention may be employed with different types of ophthalmic laser systems. A stationary system may be understood as a system that may be essentially located in a stationary manner at a single place. Similarly, a mobile system may be understood as a system that may be essentially movable, e.g. on the ground via wheels or other suitable means, to different positions or locations.

The invention further relates to an ophthalmic laser system configured to perform laser treatments on an eye of a patient using a primary beam comprising a treatment laser beam, the ophthalmic laser system comprising: a beam delivery system for focusing the primary beam so that the primary beam travels toward the eye to be treated, wherein the beam delivery system comprises a reflection prism adapted to internally deflect the primary beam toward the eye.

Thus, a compact and robust ophthalmic laser system may be obtained, as no further additional means may be necessary to deflect the primary beam towards the eye. Furthermore, the beam path of the primary beam may be suitably directed by the stationary deflection element. The deflection may be achieved by deflecting the primary beam at a certain angle from an incidence direction to an output direction.

The beam delivery system may include an objective lens system comprising one or more lenses adapted for focusing the treatment laser beam within the eye. At least a portion of the objective lens system may be part of a movable portion of the beam delivery system.

The primary beam may be created by a laser source, which may include an oscillator laser, which is configured to generate a train of low energy ultrashort pulses. A pulse energy of the low energy ultrashort pulses may be lower than 100 nJ or lower than 20 nJ, or lower than 10 nJ. The pulse energy may be greater than 1 pJ or greater than 100 pJ. The laser source may also include an amplifier, such as a regenerative amplifier or fibre amplifier for amplifying at least a portion of the low energy ultrashort pulses.

A base portion or base of the laser system may house a portion of the laser source. By way of example, the oscillator laser may be arranged in the base and the amplifier and/or a pre-compensator of the laser source, which is configured to reduce a group delay dispersion of the treatment laser beam may be arranged in the laser applicator. Alternatively, the base portion may house the entire laser source, such as the oscillator laser, the amplifier and the pre-compensator.

The base portion may include one or more housings. Each of the housings may house a portion of the laser source. By way of example, a first housing of the base may house a seed laser of the laser source and a second housing of the base may house an amplifier of the laser source, in particular a regenerative amplifier or a fibre amplifier.

The laser source may be an infrared laser source. The infrared laser source may be configured as a femtosecond laser source. A pulse energy of the laser pulses may be greater than 1 nanojoule, or greater than 10 nanojoule or greater than 50 nanojoule. The pulse energy may be less than 20 microjoule, or less than 15 microjoule or less than 10 microjoule. A pulse duration of the pulsed laser beam may be less than 800 femtoseconds, or less than 500 femtoseconds, or less than 300 femtoseconds, or less than 150 femtoseconds, or less than 100 femtoseconds. The pulse duration may be greater than 10 femtoseconds or greater than 50 femtoseconds. A repetition rate of the pulsed laser beam may be greater than 50 kHz or greater than 80 kHz. The repetition rate of the pulsed laser beam may be less than 10 MHz or less than 1 MHz. A centre wavelength of the pulsed laser beam, which is incident on the eye may be in a range of between 800 nanometers and 1400 nanometers, or between, 900 nanometers and 1400 nanometers, or between 1000 nanometers and 1100 nanometers, or between 1010 nanometers and 1050 nanometers.

The infrared laser source may be configured so that the laser pulses have a pulse energy so that the laser beam generates photodisruption within corneal tissue or within the natural lens of the patient's eye. The photodisruption may be caused by laser-induced optical breakdown. Alternatively, a pulse energy of the laser pulses may be below a threshold for generating laser-induced optical breakdown. By way of example, a plurality of pulses, which have a pulse energy below the threshold for generating laser-induced optical breakdown may be overlapped in a manner so as to generate tissue separation within the cornea.

The infrared laser source may include a pre-compensator for at least partially pre-compensating a change of the group delay dispersion (GDD) of the laser pulses, which is induced by components of the laser optical system, which are in the beam path of the laser beam downstream of the laser source. The pre-compensator may be configured to reduce the group delay dispersion. By way of example, the reduced group delay dispersion generated by the pre-compensator may have a lower positive or a more negative group delay dispersion.

A lateral diameter of the focus of the treatment laser beam of the infrared laser source within the cornea or the lens may be smaller than 10 micrometers, or smaller than 6 micrometers. The diameter may be greater than 3 micrometers. The lateral diameter may be measured in a direction perpendicular to an optical axis of the laser optical system. The lateral diameter may be measured as an 80% encircled energy diameter.

Alternatively, the laser source may be configured as an UV laser source, e.g. as an excimer laser source. The laser system may be configured to generate a laser beam, which is focused on the anterior surface of the eye. A wavelength of the treatment laser beam generated by the excimer laser source may be greater than 150 nm or greater than 190 nm. The wavelength may be less than 400 nm or less than 200 nm. A pulse duration of the treatment laser beam generated by the excimer laser source may be shorter than 100 ns or shorter than 50 ns. The pulse duration may be greater than 1 ns or greater than 3 ns.

According to a further embodiment, the reflection prism is a Bauernfeind prism.

Thus, the beam paths may be accordingly guided such that an image of the eye may be advantageously neither flipped nor rotated when being deflected.

According to a further embodiment, the reflection prism is a beam combining prism configured to combine a beam path of the primary beam and at least one secondary beam path of at least one secondary beam of an optical measuring system for acquiring measurement data of at least a portion of the eye or for illuminating at least a portion of the eye.

Thus, a reduced complexity and increase robustness of the ophthalmic laser system may be achieved. In particular, no further additional elements may be necessary to combine the primary beam and the secondary beam, e.g. to a common beam path along the output direction, as this may be achieved by the beam combining prism.

Thus, multiple beams of different systems may be employed during operation of the ophthalmic laser system and may be directed to the eye to be treated. Thus, complex surgical operations may be performed and the treatment with the primary beam may be enhanced. An optical measuring system may include any system that may allow acquiring optical data of at least a portion of the eye before, during or after the laser treatment. In some examples, measurement data of the whole eye may be acquired by the optical measuring system.

Furthermore, a compact arrangement of the ophthalmic laser system may be obtained, since no further means may be necessary to combine the primary beam with the at least one beam of the optical measuring system. A beam combination may be understood as adapting the beam paths of the primary beam and the at least one beam of the optical measuring system to travel at least partially parallel to each other or that the primary beam and the at least one beam of the optical measuring system may travel along the same beam path. In some examples, all beams of the optical measuring systems may be combined with the primary beam upstream of the stationary deflection element.

The optical measuring system may comprise an optical imaging system for acquiring a frontal image of at least a portion of the eye. Hence, the optical imaging system may acquire a frontal image of the eye to be treated, in particular an image of a region surrounding the optical axis of the beam delivery system at a location within the anterior segment of the eye. By way of example, the imaged region may include the pupil and/or at least a portion of the iris of the eye to be treated. In some examples, a frontal image of the whole eye may be acquired by the optical imaging system. The imaging beam path may extend through one or more optical elements of the patient interface, in particular through a contact element of the patient interface, which contacts an anterior surface of the cornea.

Furthermore, an additional camera may be provided, wherein the additional camera may be adapted to observe a patient's head and/or a second eye of the patient to enable a rotational compensation.

The controller of the ophthalmic laser system may include an image processing algorithm for determining, whether at least a portion of the frontal image is in focus. Additionally, or alternatively, the image processing algorithm may be configured to determine one or more parameters, which depend on, or which are indicative of, a level of focus of at least a portion of the image. The image processing algorithm may include a segmentation algorithm for segmenting the frontal image. The image processing algorithm may determine one or more parameters, which depend on, or which are indicative of, a level of focus of one or more of the segmented image regions.

The optical imaging system may acquire a frontal image of at least a portion of the eye to be treated, at least during a state in which the eye is docked or substantially docked to the ophthalmic laser system. The imaging system may include an image sensor and an imaging optical system for imaging the portion of the eye onto the image sensor. The image sensor may include a two-dimensional ordered or unordered array of pixels. The image sensor may be sensitive to one or more wavelengths within a range of between 380 nanometers and 950 nanometers or within a range of between 380 nanometers and 1400 nanometers. The imaging system may include an imaging optical system for imaging a tissue portion which is arranged in an object plane of the imaging optical system onto the image sensor. At least a portion of the imaging optical system may be provided by a portion of the beam delivery system for focusing the treatment laser beam into the eye of the patient.

A distance of an object plane of the imaging system from the laser applicator may be configured or may be adjustable so that the distance substantially corresponds to a distance of the laser applicator from the cornea of the patient's eye during the laser treatment.

The optical measuring system may further comprise a camera. Thus, a particular image information may be additionally gathered. The camera may any suitable camera, e.g. a digital camera. The camera might be connected to imaging processing means, which may allow to process acquired image data and present the image data to a user of the ophthalmic laser system. Such image data may be for instance enhanced by image processing means executing respective image processing software which may allow to manipulate the acquired image data as desired.

The optical measuring system may further comprise a confocal microscope. In one example, the treatment laser beam may be employed by the confocal microscope.

According to a further embodiment, the optical measuring system comprises an optical coherence tomography system configured for acquiring a cross-sectional image of at least a portion of the eye.

The optical coherence system and/or confocal microscope may be configured to acquire one or more cross-sectional images of the cornea and/or of at least a portion of the natural lens of the eye. The image planes of the cross-sectional images may be non-parallel to each other. The ophthalmic laser system may be configured to allow the user and/or the controller to use the images of the non-parallel image planes to align the axis of the eye to be treated with the optical axis of the movable portion of the beam delivery system.

A centre wavelength of the OCT system may be within a range of between 750 and 1400 nanometers. The optical coherence tomography system may be configured to acquire a cross-sectional image of the cornea and/or at least a portion of the natural lens of the eye. In some examples, a cross-sectional image of the whole eye may be acquired by the optical coherence tomography system.

According to a further embodiment, the OCT system includes an OCT beam scanner for scanning an OCT beam along one or more dimensions across the eye.

This may include a scanning along one or more axes in a plane, e.g. an x-y-plane, as well as a scanning along a direction perpendicular thereto, e.g. in a z-direction.

According to a further embodiment, the OCT beam scanner is separate from the scanning system of the primary beam. According to a further embodiment, the OCT beam scanner is integrated in the scanning system of the primary beam.

Thus, the OCT beam scanner may be flexibly provided in a desired separate or integrated manner.

According to a further embodiment, the ophthalmic laser system further comprises an illumination device configured for illuminating at least a portion of the eye.

Thus, portions of interest of the eye to be treated may be suitably illuminated. This may enhance the treatment abilities with the ophthalmic laser system. In some examples, the illumination device may be configured to illuminate the whole eye.

According to a further embodiment, the illumination device includes one or more light sources adapted to emit one or more secondary beams towards the reflection prism, wherein the one or more secondary beams comprise illumination light beams.

The illumination device may be configured to illuminate at least a portion of the eye. Further, a position and/or an orientation of a light emitting surface of the light source may be indicative of or correlated with an orientation of a movable portion of the beam delivery system. Additionally, or alternatively, a direction of light emitted by the light source may be indicative of or correlated with the orientation of the movable portion of the beam delivery system. The direction of light may have a fixed orientation relative to the movable portion of the beam delivery system. The orientation may be measured in a plane perpendicular to the axis of the eye to be treated.

According to a further embodiment, the illumination device comprises at least one LED light source. According to a further embodiment, the LED light source is formed as a ring extending in a plane essentially parallel to a surface of the reflection prism upon which the illumination light beams are irradiated.

Thus, a particular efficient and space-saving arrangement of the illumination device could be achieved. For instance, the illumination device may be adapted to create Purkinje images that may be employed during the operation of the laser treatment. In some examples, also more than one LED light source may be provided. In some examples, the controller may be enabled to individually switch individual LEDs or groups of LEDs and may allow to record synchronized images.

According to a further embodiment, the reflection prism is adapted to reflect the secondary beam internally twice from an incidence direction towards an output direction.

Thus, the travel direction of the beam may be advantageously directed towards the output direction via two internal reflections.

According to a further embodiment, the reflection prism has a first prism side facing the primary beam coming from a first incidence direction, a second prism side facing one or more secondary beams coming from a second incidence direction, and a third prism side facing one or more reflected beams of the one or more secondary beams reflected or back scattered by an eye of a patient along a reflected or back scattered direction.

Each of the first prism side, the second prism side and third prism side may contact the respective other two prism sides, preferably along a straight edge. In a cross-sectional view of the reflection prism, the first prism side, the second prism side in the third prism side may essentially form a triangle.

According to a further embodiment, one or more of the first prism side, the second prism side and the third prism side include reflective surfaces comprising at least partial optical coatings for reflecting the primary beam and/or secondary beam.

Thus, an enhanced reflection of the primary beam and/or the secondary beam may be achieved. The coatings may be accordingly adapted to the properties and nature of the beams employed and the one or more prism materials. The same coating may be provided for the primary beam in the secondary beam. Or different coatings may be provided, which may be adapted to the respective one of the first and/or secondary beam. As a mere example, a first coating may be provided to properly essentially reflect the primary beam while being essentially transparent to the second beam and a second coating may be provided to properly essentially reflect the second beam while being essentially transparent to the primary beam. The coatings may be accordingly adapted to reflect the respective beams totally or only partially. In some examples, one or more of the first prism side, the second prism side and the third prism side may be fully covered by respective optical coatings for reflecting the primary beam and/or the secondary beam.

According to a further embodiment, one or more of the first prism side, the second prism side and the third prism side are additionally or alternatively adapted for enabling a total internal reflection of the primary beam and/or the secondary beam.

Thus, no additional reflective surfaces may be necessary to reflect the primary beam and/or the secondary beam. The respective surfaces may be accordingly adapted to allow suitable incident and output directions for the respective beams to achieve total internal reflection.

According to a further embodiment, the optical coatings include dichroic, notch and/or partial coatings.

Thus, the optical coating may be suitably adapted to the needs of the optical system. For instance, the optical coating may be adapted to be transparent to the primary beam and to reflect the secondary beam or vice versa. Or the optical coatings may be adapted to the separate single beams included in the primary beam or the secondary beam. Furthermore, partial coatings may only cover a portion of the respective surface. Notch coatings may transmit certain wavelengths and simultaneously block or reflect others.

According to a further embodiment, the first prism side and the second prism side contact each other at a functional prism angle, wherein the functional prism angle is in the range of 22.5° to 42.5°, preferably of about 25°.

According to a further embodiment, the reflection prism adapted to guide the secondary beam along a combined beam path, wherein the secondary beam is deflected under a secondary beam deflection angle, wherein the secondary beam deflection angle is defined between the second incidence direction and the output direction, wherein the secondary beam deflection angle is in a range of 45° to 85°, preferably of about 50°.

Thus, the beam path of the secondary beam may be suitably folded or redirected from a first direction to a second direction via the reflection prism. Accordingly, devices emitting and/or receiving the secondary beam, for instance an optical measuring system and/or an OCT system may be provided flexibly in different arrangements, in particular in an inclined manner with respect to the output direction. This may allow to provide the ophthalmic laser system in a particular space-saving manner. Furthermore, the devices emitting or receiving secondary beam may be provided essentially facing a different side of the reflection prism compared to the devices emitting or receiving the primary beam.

According to a further embodiment, the reflection prism is adapted to guide the primary beam along a primary beam path, wherein the primary beam is deflected under a primary beam deflection angle, wherein the primary beam deflection angle is defined between the first incidence direction and the output direction.

Thus, the beam path of the primary beam may be suitably folded or redirected from a first direction to a second direction via the reflection prism. Accordingly, devices emitting and/or receiving the primary beam, for instance the treatment laser beam source and/or an OCT system may be provided flexibly in different arrangements, in particular in an inclined manner with respect to the output direction. This may allow to provide the ophthalmic laser system in a particular space-saving manner. Furthermore, the devices emitting or receiving the primary beam may be provided essentially facing a different side of the reflection prism compared to the devices for emitting or receiving the secondary beam.

According to a further embodiment, the functional prism angle, the secondary beam deflection angle and the primary beam deflection angle follow the relationships: β=2*α and γ=2*β.

According to a further embodiment, the second incidence direction is essentially perpendicular to the second prism side.

Thus, potential reflections at the second prism side may be mitigated and a lossless entry of the secondary beam entering the reflection prism via the second prism side may be achieved. Further, a coupling in of the secondary beam into the reflection prism may be improved. Furthermore, refractions of the secondary beam when entering the prism are avoided.

According to a further embodiment, the reflected or back scattered direction is essentially perpendicular to the third prism side or wherein the reflected or back scattered direction is inclined at an angle of up to 45° with respect to a direction perpendicular to the third prism side.

Thus, potential reflections at the third prism side may be mitigated and a lossless entry of the reflected or backscattered primary or secondary beam entering the reflection prism via the third prism side may be achieved. Thus, a coupling in of the reflected or backscattered manner primary or secondary beam into the reflection prism may be improved. Furthermore, refractions of the primary or secondary beams when entering the prism are avoided.

According to a further embodiment, the reflection prism is configured to couple the primary beam into reflection prism at the first prism side such that the primary beam travels through the reflection prism towards the second prism side and is then internally reflected at the second prism side towards the third prism side, and wherein the primary beam is then coupled out of the reflection prism at the third prism side towards the eye.

Thus, the reflection prism may provide a particular compact and space-saving beam deflection system by redirecting the primary beam path without further elements necessary. In particular, one or more, preferably each, of the beams of the primary beam may be accordingly coupled into the reflection prism at the first prism side and accordingly coupled out of the reflection prism the third prism side towards the eye.

According to a further embodiment, the primary beam is coupled out of the reflection prism essentially perpendicular to the third prism side along the output direction.

Thus, refractions of the one or more beams of the primary beam are avoided when leaving the third prism side.

According to a further embodiment, the first prism side comprises a dichroic mirroring surface covering at least partially the first prism side, wherein the dichroic mirroring surface is at least partially transparent to the primary beam and at least partially reflective to the secondary beam having one or more different optical properties than the primary beam.

Thus, the primary beam may be guided at least partially through the first prism side and at the same time the secondary beam may be at least partially reflected by the first prism side. Hence, a secondary beam may be reflected at an inner surface of the first prism side such that the secondary beam remains in the reflection prism and a primary beam may be allowed to enter the reflection prism via an opposite surface of the first prism side. Optical properties of the respective primary and secondary beams may include for instance a certain wavelength or wavelength ranges which may be accordingly different for the primary beam and the secondary beam. The primary beam may be accordingly an incoming beam that is directed onto the prism from the outside. The secondary beam may be accordingly a traversing beam that traverses the prism. In some examples, the first prism side may be fully covered by the dichroic mirroring surface. Further, in some examples, the dichroic mirroring surface may be fully transparent to the primary beam and/or fully reflective to the secondary beam.

According to a further embodiment, the reflection prism is configured to couple the secondary beam into the reflection prism at the second prism side such that the secondary beam travels through the reflection prism towards the first prism side and is reflected at the dichroic mirroring surface of the first prism side towards the second prism side, wherein the secondary beam then is internally reflected at the second prism side towards the third prism side, and wherein the secondary beam is coupled out of the reflection prism at the third prism side towards the eye.

Accordingly, a suitable beam path folding, or deflection may be obtained for the secondary beam from an incidence direction towards the second prism side to a different outcoupling direction from the third prism side towards the eye to be treated. The secondary beam may be accordingly outcoupled in the same direction as a primary beam, even though the secondary beam and the primary beam were emitted toward the prism at different prism sides.

According to a further embodiment, the outcoupled secondary beam is then at least partially reflected or backscattered by one or more features of the eye along a reflected or back scattered direction being essentially opposite to the output direction, wherein the reflection prism is configured to couple the reflected or back scattered secondary beam into the reflection prism at the third side such that the reflected or back scattered secondary beam travels through the reflection prism towards the second prism side and is then internally reflected at the second prism side towards the first prism side, wherein the reflected or back scattered secondary beam is then reflected at the dichroic mirroring surface of the first prism side towards the second prism side, and wherein the reflected or back scattered secondary beam is then coupled out of the reflection prism at the second prism side towards one or more detectors adapted to detect the reflected or back scattered secondary beam.

Thus, the reflection prism may not only allow to direct the secondary beam toward the eye but likewise allow to guide reflected or backscattered radiation of the secondary beam back to respective detectors. The beam path for the reflected backscattered radiation of the secondary beam may be accordingly the same or similar as the beam path the secondary beam travelled in the initial incidence direction, however, in a reversed direction. However, also different beam paths at the reflection prism for the reflected or backscattered secondary beam may be provided. In some examples, the outcoupled secondary beam is fully reflected backscattered by one or more features of the eye.

According to a further embodiment, the reflected or back scattered secondary beam is coupled out of the reflection prism essentially perpendicular to the second prism side.

Thus, refractions of the reflected or backscattered one or more beams of the secondary beam are avoided when leaving the prism at the second prism side.

According to a further embodiment, the primary beam is coupled into the reflection prism at the first prism side at an angle in a range of 80° to 100°, preferably 85° to 95°, more preferably 82° to 92°.

According to a further embodiment, the primary beam is internally reflected at the second prism side at an angle in a range of larger than 90° to 170°, preferably of about 100°.

According to a further embodiment, the secondary beam and/or the reflected or back scattered secondary beam is reflected at the dichroic mirroring surface of the first prism side at an angle in a range of 45° to 85°, preferably of about 50°.

According to a further embodiment, the secondary beam and/or the reflected or back scattered secondary beam is internally reflected at the second prism side at an angle in a range of larger than 90° to 170°, preferably of about 100°.

The invention further relates to an ophthalmic laser system configured to perform laser treatments on an eye of a patient using a primary beam, the ophthalmic laser system comprising: a truncated cone-shaped optical element for guiding a primary beam of the ophthalmic laser system toward the eye, wherein the truncated cone-shaped optical element is arranged between the eye and a stationary deflection element adapted to deflect the primary beam at a deflection angle toward the eye, wherein the truncated cone-shaped optical element tapers along a second optical axis towards the eye.

Thus, the primary beam may be guided along the extension direction, for instance parallel to or along a rotation axis of the truncated cone-shaped optical element from the stationary deflection element towards the eye. Thus, a defined beam path along the truncated cone-shaped optical element having defined optical properties may be provided, which may facilitate the guidance of the primary beam toward the eye to be treated.

The primary beam may be created by a laser source, which may include an oscillator laser, which is configured to generate a train of low energy ultrashort pulses. A pulse energy of the low energy ultrashort pulses may be lower than 100 nJ or lower than 20 nJ, or lower than 10 nJ. The pulse energy may be greater than 1 pJ or greater than 100 pJ. The laser source may also include an amplifier, such as a regenerative amplifier or fibre amplifier for amplifying at least a portion of the low energy ultrashort pulses.

A base portion or base of the laser system may house a portion of the laser source. By way of example, the oscillator laser may be arranged in the base and the amplifier and/or a pre-compensator of the laser source, which is configured to reduce a group delay dispersion of the treatment laser beam may be arranged in the laser applicator. Alternatively, the base portion may house the entire laser source, such as the oscillator laser, the amplifier and the pre-compensator.

The base portion may include one or more housings. Each of the housings may house a portion of the laser source. By way of example, a first housing of the base may house a seed laser of the laser source and a second housing of the base may house an amplifier of the laser source, in particular a regenerative amplifier or a fibre amplifier.

The laser source may be an infrared laser source. The infrared laser source may be configured as a femtosecond laser source. A pulse energy of the laser pulses may be greater than 1 nanojoule, or greater than 10 nanojoule or greater than 50 nanojoule. The pulse energy may be less than 20 microjoule, or less than 15 microjoule or less than 10 microjoule. A pulse duration of the pulsed laser beam may be less than 800 femtoseconds, or less than 500 femtoseconds, or less than 300 femtoseconds, or less than 150 femtoseconds, or less than 100 femtoseconds. The pulse duration may be greater than 10 femtoseconds or greater than 50 femtoseconds. A repetition rate of the pulsed laser beam may be greater than 50 kHz or greater than 80 kHz. The repetition rate of the pulsed laser beam may be less than 10 MHz or less than 1 MHz. A centre wavelength of the pulsed laser beam, which is incident on the eye may be in a range of between 800 nanometers and 1400 nanometers, or between, 900 nanometers and 1400 nanometers, or between 1000 nanometers and 1100 nanometers, or between 1010 nanometers and 1050 nanometers.

The infrared laser source may be configured so that the laser pulses have a pulse energy so that the laser beam generates photodisruption within corneal tissue or within the natural lens of the patient's eye. The photodisruption may be caused by laser-induced optical breakdown. Alternatively, a pulse energy of the laser pulses may be below a threshold for generating laser-induced optical breakdown. By way of example, a plurality of pulses, which have a pulse energy below the threshold for generating laser-induced optical breakdown may be overlapped in a manner so as to generate tissue separation within the cornea.

The infrared laser source may include a pre-compensator for at least partially pre-compensating a change of the group delay dispersion (GDD) of the laser pulses, which is induced by components of the laser optical system, which are in the beam path of the laser beam downstream of the laser source. The pre-compensator may be configured to reduce the group delay dispersion. By way of example, the reduced group delay dispersion generated by the pre-compensator may have a lower positive or a more negative group delay dispersion.

A lateral diameter of the focus of the treatment laser beam of the infrared laser source within the cornea or the lens may be smaller than 10 micrometers, or smaller than 6 micrometers. The diameter may be greater than 3 micrometers. The lateral diameter may be measured in a direction perpendicular to an optical axis of the laser optical system. The lateral diameter may be measured as an 80% encircled energy diameter.

Alternatively, the laser source may be configured as an UV laser source, e.g. as an excimer laser source. The laser system may be configured to generate a laser beam, which is focused on the anterior surface of the eye. A wavelength of the treatment laser beam generated by the excimer laser source may be greater than 150 nm or greater than 190 nm. The wavelength may be less than 400 nm or less than 200 nm. A pulse duration of the treatment laser beam generated by the excimer laser source may be shorter than 100 ns or shorter than 50 ns. The pulse duration may be greater than 1 ns or greater than 3 ns.

The stationary deflection element may be understood as an element that may enable a deflection of the primary beam, which is irradiated to the stationary deflection element at a certain angle, toward the eye at a fixed output angle. “Stationary” in this context may be understood such that the stationary deflection element is essentially not moved or tiled during the laser treatment. Of course, the output angle may vary, e.g. during conductance of the laser treatment beam, depending on the input angle of the primary beam. Nevertheless, the stationary deflection element may of course be moved to find a suitable position or orientation with respect to an eye to be treated.

According to a further embodiment the ophthalmic laser system comprises an optical measuring system for acquiring measurement data of at least a portion of the eye or for illuminating at least a portion of the eye.

An optical measuring system may include any system that may allow acquiring optical data of at least a portion of the eye before, during or after the laser treatment. In some examples, measurement data of the whole eye may be acquired by the optical measuring system.

In some examples, the optical measuring system may comprise an optical coherence tomography system configured for acquiring a cross-sectional image of at least a portion of the eye.

A centre wavelength of the OCT system may be within a range of between 750 and 1400 nanometers. The optical coherence tomography system may be configured to acquire a cross-sectional image of the cornea and/or at least a portion of the natural lens of the eye. In some examples, a cross-sectional image of the whole eye may be acquired by the optical coherence tomography system.

In some examples the OCT system may include an OCT beam scanner for scanning an OCT beam along one or more dimensions across the eye.

This may include a scanning along one or more axes in a plane, e.g. an x-y-plane, as well as a scanning along a direction perpendicular thereto, e.g. in a z-direction.

In some examples, the OCT beam scanner may be separate from the scanning system of the primary beam. In some examples, the OCT beam scanner may be integrated in the scanning system of the primary beam.

Thus, the OCT beam scanner may be flexibly provided in a desired separate or integrated manner.

In some examples, the optical measuring system may comprise an optical imaging system for acquiring a frontal image of at least a portion of the eye.

The optical imaging system may acquire a frontal image of the eye to be treated, in particular an image of a region surrounding the optical axis of the beam delivery system at a location within the anterior segment of the eye. By way of example, the imaged region may include the pupil and/or at least a portion of the iris of the eye to be treated. In some examples, a frontal image of the whole eye may be acquired by the optical imaging system. The imaging beam path may extend through one or more optical elements of the patient interface, in particular through a contact element of the patient interface, which may contact an anterior surface of the cornea.

Furthermore, an additional camera may be provided, wherein the additional camera may be adapted to observe a patient's head and/or a second eye of the patient to enable a rotational compensation.

A controller of the ophthalmic laser system may include an image processing algorithm for determining, whether at least a portion of the frontal image is in focus. Additionally, or alternatively, the image processing algorithm may be configured to determine one or more parameters, which depend on, or which are indicative of, a level of focus of at least a portion of the image. The image processing algorithm may include a segmentation algorithm for segmenting the frontal image. The image processing algorithm may determine one or more parameters, which depend on, or which are indicative of, a level of focus of one or more of the segmented image regions.

The optical imaging system may acquire a frontal image of at least a portion of the eye to be treated, at least during a state in which the eye is docked or substantially docked to the ophthalmic laser system. The imaging system may include an image sensor and an imaging optical system for imaging the portion of the eye onto the image sensor. The image sensor may include a two-dimensional ordered or unordered array of pixels. The image sensor may be sensitive to one or more wavelengths within a range of between 380 nanometers and 950 nanometers or within a range of between 380 nanometers and 1400 nanometers. The imaging system may include an imaging optical system for imaging a tissue portion which is arranged in an object plane of the imaging optical system onto the image sensor. At least a portion of the imaging optical system may be provided by a portion of the beam delivery system for focusing the treatment laser beam into the eye of the patient.

A distance of an object plane of the imaging system from the laser applicator may be configured, or may be adjustable so that, the distance substantially corresponds to a distance of the laser applicator from the cornea of the patient's eye during the laser treatment.

In some examples, the optical measuring system may further comprise a camera.

Thus, a particular image information may be additionally gathered. The camera may any suitable camera, e.g. a digital camera. The camera might be connected to imaging processing means, which may allow to process acquired image data and present the image data to a user of the ophthalmic laser system. Such image data may be for instance enhanced by the image processing means executing respective image processing software which may allow to manipulate the acquired image data as desired.

In some examples, the optical measuring system may further comprise a confocal microscope.

The optical coherence system and/or confocal microscope may be configured to acquire one or more cross-sectional images of the cornea and/or of at least a portion of the natural lens of the eye. The image planes of the cross-sectional images may be non-parallel to each other. The ophthalmic laser system may be configured to allow the user and/or the controller to use the images of the non-parallel image planes to align the axis of the eye to be treated with the optical axis of the movable portion of the beam delivery system. In one example, the treatment laser beam may be employed by the confocal microscope.

According to a further embodiment, the stationary deflection element is adapted to combine one or more secondary beams of the optical measuring system with the primary beam, and wherein the truncated cone-shaped optical element is adapted to guide the combined primary beam and the one or more secondary beams towards the eye.

Thus, also the secondary beam may be guided along the extension direction, for instance parallel to or along a rotation axis of the truncated cone-shaped optical element from the stationary deflection element towards the eye. Thus, a defined beam path along the truncated cone-shaped optical element having defined optical properties may be provided, which may facilitate the guidance of the secondary beam toward the eye to be treated.

Further, a compact arrangement of the ophthalmic laser system could be obtained, since no further means are necessary to combine the primary beam with the secondary beam. A beam combination may be understood as adapting the beam paths of the primary beam and the at least one beam of the secondary beam to travel at least partially parallel to each other or that the primary beam and the secondary beam may travel along the same beam path. In some examples, all beams of the optical measuring systems may be combined with the primary beam upstream of the stationary deflection element.

According to a further embodiment, the stationary deflection element is a reflection prism, comprising a first prism side facing the primary beam coming from a first incidence direction, a second prism side facing the one or more secondary beams coming from the second incidence direction, and a third prism side facing one or more reflected beams of the one or more secondary beams reflected or backscattered by the eye along a reflected or backscattered direction, wherein the truncated cone-shaped optical element comprises an essentially flat first cone end surface facing the third prism side and an oppositely arranged second cone end surface facing the eye.

Thus, a reduced complexity and increased robustness of the ophthalmic laser system may be achieved. In particular, no further additional elements might be necessary to combine the primary beam and the secondary beam to a common beam path along the output direction, as this may be achieved by the reflection prism.

Further, the coupling of the primary beam and/or the secondary beam from the reflection prism to the truncated cone-shaped optical element and vice versa via the interface at the essentially flat first cone end surface and the third prism side may be provided. Likewise, an outcoupling of the primary beam and/or the secondary beam from the second cone end surface toward the eye may be enabled. The essentially flat first cone end surface may not be construed being fully flat, but they may also be provided with a minor curvature to provide optimized optical properties for in- and outcoupling the primary beam and/or the secondary beam from and into the truncated cone-shaped optical element, respectively. Furthermore, also optimized treatment ranges and precisions in the positioning of the primary beam and/or the secondary beam in the eye to be treated may be considered by the shape of the essentially flat first cone end surface and the second cone end surface.

According to a further embodiment, the reflection prism is a Bauernfeind prism.

Thus, the beam paths may be accordingly guided such that an image of the eye may be advantageously neither flipped nor rotated when being deflected.

According to a further embodiment, the reflection prism is a beam combining prism configured to combine a beam path of the primary beam and at least one secondary beam path of at least one secondary beam of the optical measuring system.

Thus, a reduced complexity and increase robustness of the ophthalmic laser system may be achieved. In particular, no further additional elements may be necessary to combine the primary beam at least one secondary beam of the optical measuring system to a common beam path, as this may be achieved by the beam combining prism.

According to a further embodiment, the essentially flat first cone end surface contacts at least partially the third prism side.

Thus, a predefined interface between the reflection prism and the truncated cone-shaped optical element may be obtained by a contact between the essentially flat first cone end surface and the third prism side. A contact may be either direct contact between the essentially flat first cone end surface and the third prism side or may be also an indirect contact with further elements arranged in between the first cone end surface and the third prism side. In some examples, the essentially flat end surface may fully contact the third prism side.

According to a further embodiment, the ophthalmic laser system further comprises a patient interface arranged between the second cone end surface and the eye for docking the eye to the ophthalmic laser system.

Thus, an improved guidance of the primary and secondary beams into and out of the eye to be treated may be achieved. During the laser treatment, the patient's eye may be docked to the portion of the beam delivery system e.g. via a contact element of a patient interface, wherein the contact element may contact an anterior surface of the cornea of the patient's eye and the treatment laser may traverse the contact element after having exited the beam delivery system. The docking may be provided as a reversible mechanical connection between the eye and the ophthalmic laser system.

According to a further embodiment, the truncated cone-shaped optical element is adapted to provide the primary beam to the eye at a numerical aperture of 0.1 to 1.0, preferably of 0.15 to 0.6, more preferably of 0.15 to 0.4 and most preferred of 0.18 to 0.25 is provided.

The present invention further relates to a method for operating an ophthalmic laser system. The method comprises the step of providing a beam delivery system for focusing the primary beam so that the primary beam travels toward the eye to be treated. The method further comprises the step of providing a scanning system for scanning a focus of the primary beam within the eye or on an anterior surface of the eye in two or more dimensions. The beam delivery system comprises a stationary deflection element adapted to deflect the primary beam at a deflection angle toward the eye, wherein the deflection angle is defined as an angle between: an incident portion of the primary beam along a first optical axis of one or more upstream optical elements of the beam delivery system, wherein the upstream optical elements are arranged in a beam path of the primary beam upstream of the stationary deflection element; and a deflected portion of the primary beam along a second optical axis of one or more downstream optical elements of the beam delivery system, wherein the downstream optical elements are arranged in the beam path of the primary beam downstream of the stationary deflection element. The method further comprises the step of deflecting primary beam at angle larger than 90°.

Hence, an improved method for operating an ophthalmic laser system may be provided, which may enable a space efficient operation of the ophthalmic laser system. The ophthalmic laser system may also be arrangeable in a more flexible manner.

The present invention further relates to a method for operating an ophthalmic laser system, wherein the method comprises the steps of: providing a beam delivery system for focusing the primary beam so that the primary beam travels toward an eye to be treated, providing a reflection prism adapted to internally deflect the primary beam toward the eye, and irradiating the primary beam toward the reflection prism, wherein the primary beam is internally deflected from the reflection prism toward the eye to be treated.

Thus, a facilitated operation of the ophthalmic laser system may be achieved, as no further additional means may be necessary to deflect the primary beam towards the eye. Furthermore, the beam path of the primary beam may be suitably directed by the reflection prism. The deflection may be achieved by deflecting the primary beam at a certain angle towards the eye to be treated.

The present invention further relates to a method for operating an ophthalmic laser system comprising the step of providing a truncated cone-shaped optical element. The method further comprises the step of arranging the truncated cone-shaped optical element between the eye and a stationary deflection element adapted to deflect the primary beam at a deflection angle toward the eye, wherein the truncated cone-shaped optical element tapers along a second optical axis towards the eye. The method further comprises the step of guiding the primary beam of the ophthalmic laser system from the deflection element via the truncated cone-shaped optical element to the eye to be treated.

Thus, an improved operation of the ophthalmic laser system may be provided including a facilitated guidance of the primary beam toward the eye to be treated.

Regarding the individual features of the ophthalmic laser system described with respect to the above method steps, it is referred to the detailed explanations of said features described with respect to the ophthalmic laser system, which accordingly apply.

It is clear for a skilled person that any aspects described above in connection with one embodiment of the present invention may be readily employed also in combination with other embodiments of the present invention for which said aspects may not be explicitly described. Further features, examples, and advantages will become apparent from the following detailed description of preferred embodiments and the accompanying figures.

### BRIEF DESCRIPTION OF THE DRAWINGS

For a better understanding of the present invention, and to illustrate its practicality, figures are provided in the following and reference is made thereto. It should be understood that the figures represent only exemplary embodiments and thus in no way limit the scope of the claimed invention. Identical or like-acting elements are indicated throughout by the same reference signs. Hence, any explanation with respect to a certain element or feature in one figure apply likewise also to the same or similar element shown in a different figure. Sometimes, reference signs for certain elements have been omitted for the sake of representability. However, respective explanations of said elements made in another figure apply accordingly to the element not explicitly indicated. Any reference signs in the claims should not be construed as limiting the scope of the claims. The figures are merely schematic representations and serve only to illustrate examples of the disclosure. Further, as used herein, a hyphenated form of a reference sign refers to a specific embodiment of an element and the unhyphenated form of the reference numeral refers to the collective element. For example, a device “59-1” refers to an instance of a device class that can be collectively referred to as device “59” and each of which can be generically referred to as device “59”.

In the accompanying drawings:

FIG. 1 is a perspective view of a laser system according to an exemplary embodiment.

FIG. 2 is a second perspective view of the laser system according to the exemplary embodiment, which is shown in FIG. 1 wherein the support arm is in a resting position.

FIG. 3 is a schematic view of the laser system of the exemplary embodiment which is shown in FIG. 1 and which illustrates the extent of a lateral movement range of the laser applicator of the laser system according to the exemplary embodiment, which is illustrated in FIG. 1;

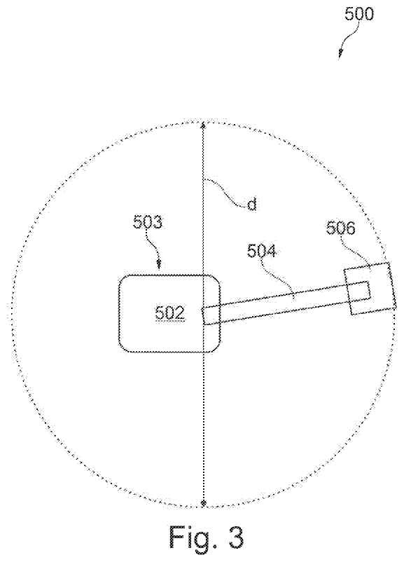

FIG. 4 is a further schematic top view of the laser system according to the exemplary embodiment which is shown in FIG. 1;

FIG. 5 is a schematic side view of the support arm and the laser applicator of the laser system according to the exemplary embodiment, which is shown in FIG. 1;

FIG. 6 is a schematic view of information shown on a display device of the laser applicator of the laser system according to the exemplary embodiment, which is shown in FIG. 1;

FIG. 7 is a schematic view of an imaging system, an OCT system and beam combiners which are arranged in the laser applicator of the laser system according to the exemplary embodiment, which is shown in FIG. 1.

FIG. 8 is a schematic view of a parallel linkage and a counterbalancing mechanism of the support arm of the laser system according to the exemplary embodiment, which is illustrated in FIG. 1; and

FIGS. 9A to 9C are side views of the support arm and an articulated beam guide tube of the laser system and a coupling arrangement for coupling the articulated beam guide tube to the support arm of the laser system according to the exemplary embodiment.

FIG. 10 illustrates a schematic cross-sectional view of an ophthalmic laser system according to an exemplary embodiment of the present invention;

FIG. 11 illustrates a schematic cross-sectional view of an ophthalmic laser system according to an exemplary embodiment of the present invention;

FIG. 12 illustrates a schematic cross-sectional view of an ophthalmic laser system according to an exemplary embodiment of the present invention;

FIG. 13 illustrates a schematic cross-sectional view of an ophthalmic laser system according to an exemplary embodiment of the present invention;

FIG. 14 illustrates an enlarged cross-sectional view of a stationary deflection element of an ophthalmic laser system according to an exemplary embodiment of the present invention;

FIG. 15A to 15C illustrate schematic flowcharts of methods for operating an ophthalmic laser system according to an exemplary embodiment of the present invention.

### DETAILED DESCRIPTION OF EXEMPLARY EMBODIMENTS

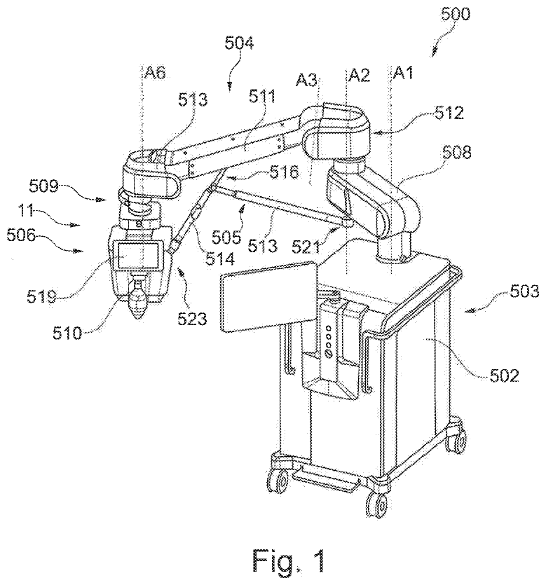

FIG. 1 is a schematic illustration of an ophthalmic laser system 500 according to an exemplary embodiment for performing laser treatments of an eye. The treatments may include, but are not limited to, forming flaps for laser assisted in situ keratomileusis (LASIK) treatments, forming corneal incisions and limbal relaxing incisions (LRI, AK), performing capsulotomy (in particular anterior capsulotomy) and lens fragmentation.

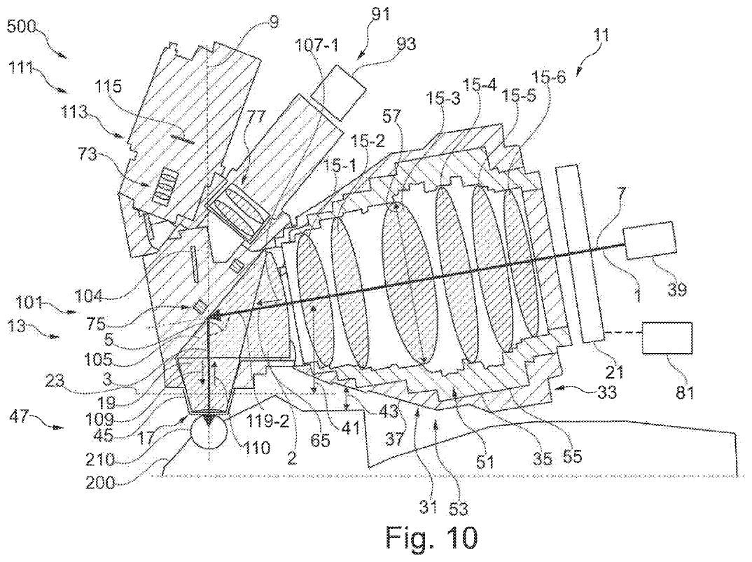

The laser system 500 may be for instance a stationary system or a mobile system and includes a laser source 39 (not depicted here, see e.g. FIGS. 10-13), which is configured for generating a primary beam which may include or consist of a treatment laser beam for performing the laser treatments. At least a portion of the laser source 39 is mounted within a housing 502 of a base portion or base 503. A further portion of the laser source 39 may be arranged within a laser applicator 506, whereby the laser applicator 506 is supported by an articulated support arm 504. Alternatively, the further portion of the laser source 39 may also be partially arranged within the articulated support arm 504 supporting the laser applicator 506, or further, be arranged entirely within the articulated support arm 504 which supports the laser applicator 506. For brevity, the term ‘articulated support arm 504’ may also be referred to as ‘support arm 504’. Both terms being used interchangeably throughout the body of the specification without prejudice.

It is also conceivable that the base portion 503 includes more than one housing 502, wherein each housing houses a portion of the laser source 39. By way of example, a first housing of the base portion 503 houses an oscillator laser of the laser source 39 and a second housing of the base portion 503 houses an amplifier and/or a pre-compensator of the laser source 39.

In the exemplary embodiment shown in FIG. 1, the laser source 39 is arranged in the base portion 503. However, as discussed above it is also conceivable that only a first portion of the laser source 39 is arranged in the base portion 503 and a second portion of the laser source 39 is arranged in the laser applicator 506 or in the support arm 504. By way of example, an oscillator laser of the laser source 39 may be arranged in the base portion 503 and the regenerative amplifier and/or a pre-compensator of the laser source 39 may be arranged in the laser applicator 506 and/or support arm 504.

The laser system 500 comprises a beam delivery system 11, which also may be referred to as a laser optical system, is configured to direct a laser beam towards the eye 210 of a patient 200 (not depicted here, see e.g. FIGS. 10-13) who is disposed on a patient bed or support, which is not illustrated in FIG. 1. The laser source 39 generates a treatment laser beam, which is guided by the laser optical system 11 through a portion of a support arm 504, an articulated beam guide tube 505, and a laser applicator 506. The articulated beam guide tube 505 extends at least between a first location 521, where the laser beam exits from the support arm 504 and a second location 523, where the laser beam enters into the laser applicator 506. However, the present disclosure is not limited to such a configuration for guiding the treatment laser beam from the base portion 503 to the patient's eye. By way of example, in another embodiment, the laser beam may be guided through the entire support arm 504. In such an embodiment, the laser system 500 may be configured so that it does not include a beam guide tube 505. It is also conceivable that the articulated beam guide tube 505 extends at least between a first location, where the treatment laser beam exits from the base portion 503 to a second location, where the treatment laser beam enters into the support arm 504 or into the laser applicator 506. A portion of the articulated beam guide tube 505 may be arranged within the base portion 503, within the support arm 504 and/or within the laser applicator 506.

In the exemplary embodiment, which is shown in FIG. 1, the laser beam source 39 is configured to emit a pulsed laser beam having a pulse energy and a pulse duration, which is sufficient to generate laser-induced optical breakdown (LIOB) within the cornea or the natural lens of the patient's eye 210. The laser-induced optical breakdown generated by a laser pulse leads to photodisruption, so that a series of consecutive overlapping or closely located laser pulses generate cuts within the corneal tissue or the natural lens. Photodisruption is a nonthermal process. The laser optical system 500 includes a scanning system 21 (not depicted here, see e.g. FIGS. 10-13), which is configured to scan a focus of the pulsed laser beam within the eye 210 to form perforated cuts or continuous (i.e. non-perforated) cuts. In the exemplary embodiment, which is shown in FIG. 1, the scanning system 21 is arranged within the laser applicator 506. However, it is also conceivable that at least a portion of the scanning system 21 is arranged within the base portion 503 and/or within the support arm 504.

It is noted that the present disclosure is not limited to the above laser treatments and laser systems. Specifically, the laser system 500 may be configured to controllably ablate corneal tissue without causing significant damage to adjacent and/or underlying tissues of the eye. The laser system 500 may emit light having a wavelength greater than 150 nm or greater than 190 nm. The wavelength may be less than 400 nm or less than 200 nm. By way of example, the laser source 39 may be configured as an excimer laser source. The laser system 500 may be an argon-fluorine excimer laser, which generates pulses of laser light having a wavelength of substantially 193 nm. The laser ablation process may be used for reshaping the cornea. Such ablative treatments may include, but are not limited to, photorefractive keratectomy, laser assisted subepithelial keratomileusis (LASEK), laser-assisted in-situ keratomileusis (LASIK) and phototherapeutic keratectomy. In each of these procedures, the laser beam may be used to remove a predetermined amount of the corneal stroma which is located beneath the corneal epithelium and Bowman's membrane to form a reshaped surface portion.

Using the support arm 504, the laser applicator 506 is positionable in three dimensions. The support arm 504 may be configured to allow manual positioning of the laser applicator 506 in three dimensions. However, it is also conceivable that the support arm 504 may include one or more motors so that at least a portion of the movements, which can be carried out using the support arm 504, are motorized. In the embodiment shown, a three-axis positioning system 509 is arranged between the support arm 504 and the laser applicator 506. In this embodiment, the three-axis positioning system 509 is motorized. The three-axis positioning system 509 may be configured such that the laser applicator 506 is positionable relative to the support arm 504. In an alternate embodiment, the positioning system 509 is part of the support arm 504 such that the laser applicator 506 and a portion of the support arm 504 is positionable relative to a further portion of the support arm 504 or relative to the base portion 503. By way of example, the positioning system 509 may be arranged between first and second arm segments 508, 511 or may be part of an arm segment of the support arm 504. In one example, the positioning system 509 may be implemented between the first arm segment 508 and the housing 502.

By way of example, as is described in more detail below, the positioning system 509 is used for fine positioning procedure after the laser applicator 506 has been positioned in a course positioning procedure using the support arm 504.

The positioning system 509 may be operatively coupled to a controller (not shown in FIG. 1). The controller may be arranged within the base portion 503. However, it is also conceivable that the controller is arranged in a housing which is separate from the base portion 503 and also separate from the laser applicator 506. The controller may be connected to the base portion 503, to the positioning system 509 and/or components of the laser applicator 506 via a wired or wireless connection.

The laser applicator 506 may further include a manually operable control unit 510, which is operatively coupled to the controller for performing the positioning of the laser applicator 506 relative to the portion or relative to the entire support arm 504 using the positioning system 509. The manually operable control unit 510 may be configured for directional control, in particular for directional control in three dimensions. By way of example, the control unit 510 may be configured as a joystick (such as illustrated in FIG. 1) and/or may include one or more buttons. Each of the button may correspond to a direction of travel. However, the present disclosure is not limited to such a control element. It is also conceivable that the control element includes one or more buttons, each of which representing a direction of travel of the laser applicator 506.

The configuration of the ophthalmic laser system 500 according to the exemplary embodiment allows positioning the laser applicator 506 relative to the patient 200, so that the patient 200 can remain in the regular patient bed where the patient 200 has received pre-surgical treatment. Thereby, patient transfers can be avoided, which otherwise would be required for moving patients from their regular patient beds to a separate stationary patient support specifically provided for conducting the laser surgical steps.

For this reason, the surgical ophthalmic laser system 500 according to the present disclosure not only increases the effectiveness of the clinical workflow but also reduces the footprint required in the surgical operation theatre, since there is no space necessary for a fixedly installed patient support, which is provided solely for performing the laser surgery. Further, a movable laser surgical system facilitates cleaning and sterilization of the surgical operation theatre.

Moreover, in order to reduce footprint and costs, it is desirable that femtosecond laser systems, which are configured for cataract surgery also be used for forming corneal flaps for laser in situ keratomileusis (LASIK). A femtosecond laser for forming LASIK flaps, however, should be located close to the excimer laser system in order to make it easy for the patient to move from one system to the other. On the other hand, a femtosecond laser for cataract surgery, should be located in the sterile operating theatre, where the natural lens is emulsified using a phacoemulsification device and where the intraocular lens is implanted. The laser system 500 according to the exemplary embodiment is a mobile laser system, which can easily be moved between different locations in the hospital. Also, the articulated support arm allows the surgeon to more flexibly arrange the laser system 500 within the surgical operating room, where many other devices, such as a surgical microscope, a phacoemulsification system and operating room trolleys are located and where there must be sufficient space for one or more surgeons and other medical staff members.

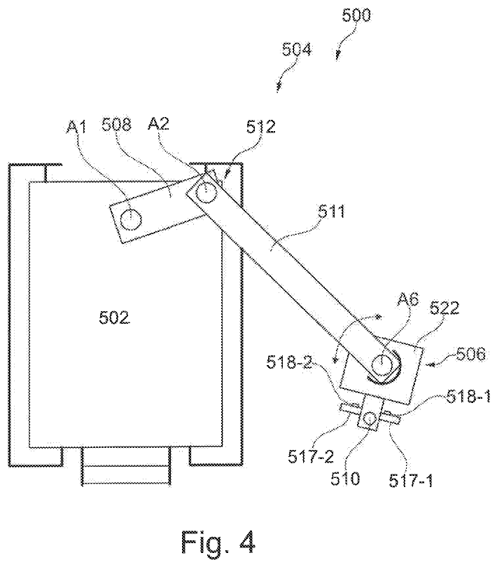

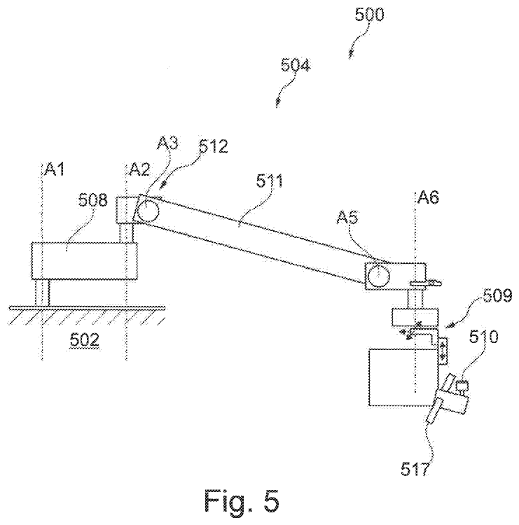

As is illustrated in FIG. 1 and also in the schematic top view of FIG. 4 and the schematic side view of FIG. 5, the support arm 504 may be configured as an articulated support arm having two or more arm segments, which are connected in series. The support arm 504 may include a first arm segment 508 and a second arm segment 511. The first arm segment 508 may be rotatably supported by the base portion 503 so that the first arm segment 508 is rotatable about a first vertical axis A1. The first vertical axis A1 may have a fixed position and orientation relative to the base portion 503. Additionally, or alternatively, the base may be configured so that position, in particular a height, inclination and/or orientation (rotation) of the first arm segment 508 relative to the base portion 503 is adjustable. By way of example, the base portion 503 may be configured so that the first arm segment 508 is supported by the base portion 503 using a rotational bearing assembly (not shown in FIG. 1) for rotatably supporting the first arm segment 508 about the first vertical axis A1. The base portion 503 may be configured so that a height of the rotational bearing assembly relative to remaining portions of the base portion 503 is adjustable, e.g. using a motorized heigh adjusting mechanism.

The first arm segment 508 may be connected to the second arm segment 511 via an intermediate joint 512. The intermediate joint 512 may be configured so that an orientation of the second arm segment 511 relative to the first arm segment 508 is adjustable in two dimensions. By way of example, the intermediate joint 512 may be configured so that the second arm segment is rotatable about a vertical axis A2 and also rotatable about a horizontal axis A3.

The support arm 504 is configured so that before and after a rotation of the second arm segment 511 about the horizontal axis A3, the laser applicator 506 has a same vertical orientation. In the exemplary embodiment, the rotation of the second arm segment 511 about the horizontal axis A3 is coupled with an orientation of the laser applicator 506 relative to the second arm segment 511. The coupling is a mechanical coupling which is obtained through a multi bar linkage of the support arm 504, which may be configured as a parallel linkage. The multi bar linkage may be configured so that an orientation of a first end of the multi bar linkage relative to a second end of the multi bar linkage is maintained while moving the first end relative to the second end. The multi bar linkage may be a parallel linkage and/or a four-bar linkage. The multi bar linkage may be balanced in order to provide at least partial gravity counterbalancing for the laser applicator 506. Two or more parallel bars of the parallel linkage may form the second arm segment 511. However, it is also conceivable that the second arm segment 511 and/or the laser applicator 506 include an inclination sensor for measuring an inclination of the second arm segment 511 and/or the laser applicator 506 relative to the horizontal plane. A controller may be provided which receives output signals from the one or more inclination sensors and which controls an adjustment of the vertical orientation of the laser applicator 506 based on the measured inclination. The adjustment of the vertical orientation may be performed using a motor which drives a rotational joint. The arm segments 508, 511 are connected in series via rotational joints 548. Each of the rotational joints 548 may be configured so that an orientation of neighbouring arm segments relative to each other is adjustable. Each of the rotational joint 548 may have either one or two rotation axes. The support arm 504 may include at least one multi-axis joint, which has two or more rotation axes. Each of the rotation axes may be oriented substantially orthogonal or orthogonal relative to each other. One of the two rotation axes may be substantially oriented or oriented along a vertical direction.

It is also conceivable that the connection between the second arm segment 511 and the laser applicator 506 is configured so that the vertical orientation of the laser applicator 506 is maintained by gravitational forces acting on the laser applicator 506.

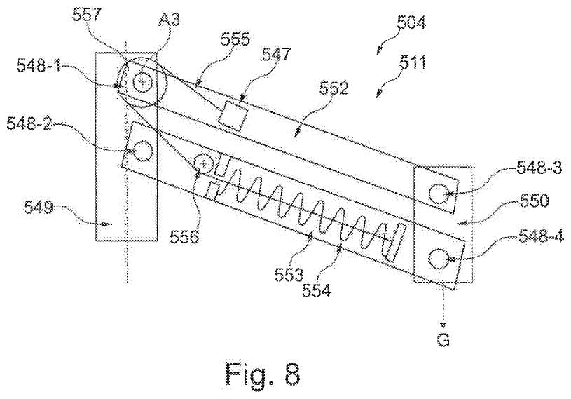

The parallel linkage may be configured so that a distal end of parallel linkage (i.e. distal relative to the base portion 503) maintains its vertical orientation, irrespective of the orientation of the second arm segment 511 relative to the horizontal plane. An example for a parallel linkage is described below with reference to FIG. 8.

As can be seen from FIGS. 1, 4 and 5, the laser applicator 506 can be rotated relative to the support arm 504 about a horizontal rotation axis A5 and a vertical rotation axis A6. The vertical rotation axis A6 may extend through the laser applicator 506, in particular through a housing of the laser applicator 506. However, it is also conceivable that the vertical rotation axis A6 does not extend through the laser applicator 506. Rotating the laser applicator about the vertical rotation axis A6 changes an orientation of the laser applicator 506 relative to the support arm 504.

Rotating the laser applicator 506 relative to at least a portion of the support arm 504 allows the surgeon during the coarse positioning procedure to adjust the orientation of the laser applicator 506 so that the positioning of the laser applicator 506 relative to the patient's eye 210 is not hindered by space constraints defined by the patient's anatomy.

The laser system 500 according to the exemplary embodiment includes a braking and/or locking system for arresting a movement of the second end of the support arm 504 relative to the first end of the support arm 504. In one example, the braking and/or locking system may enable to brake and/or lock a movement around the vertical axis A2. Specifically, this allows the surgeon to perform the coarse positioning procedure by manually positioning the laser applicator 506 into a position close to the patient's head. After the coarse positioning procedure, the surgeon activates a braking and/or locking system of the support arm 504 so that in the arrested state, the surgeon can perform the fine positioning procedure using the positioning system 509.

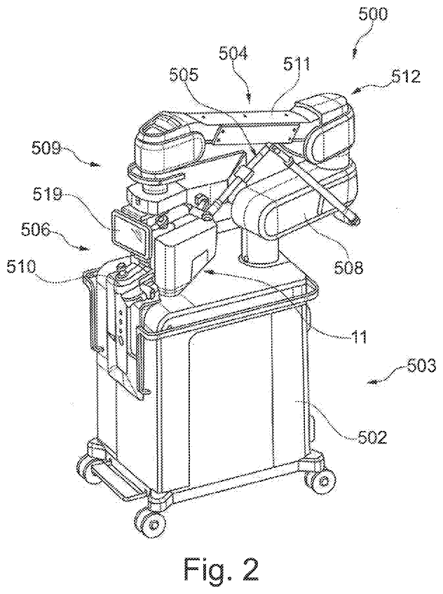

In the exemplary embodiment, the laser applicator 506 includes one or more manually operable control elements 518-1, 518-2, which allow the surgeon to deactivate the braking and/or locking system so that the applicator head can be positioned to a different position using the support arm 504. In the exemplary embodiment, the laser applicator 506 includes two handles 517, indicated separately as 517-1, 517-2 shown in FIG. 4, which can be grasped by both hands of the surgeon. At each of the handles a release button 518-1, 518-2 is provided. If the surgeon simultaneously presses both release buttons, the braking and/or locking system is deactivated and the surgeon can position the laser applicator 506 in three dimensions. For simplicity of illustration, in FIGS. 1 and 2, the handles 517-1, 517-2 and the release buttons 518-1 and 518-2 are not shown.

The laser system 500 according to the exemplary embodiment is configured to allow positioning the laser applicator 506 relative to the patient's head by performing a coarse positioning procedure in which the laser applicator 506 is adjusted relative to the patient's head using the support arm 504. Then, in a subsequent fine positioning procedure using the three-axis positioning system 509, the laser applicator 506 is positioned to its final position relative to the patient's eye in which the laser treatment is carried out.

The coarse positioning procedure using the support arm 504 allows the surgeon to perform a fast and efficient coarse positioning relative to the patient's head. The manual adjustability also provides increased safety for the patient, since the surgeon can rapidly move the laser applicator 506 to a location distant from the patient's head if needed. However, it is conceivable that one or more joints of the support arm 504 are motorized so that the coarse positioning is performed fully or partially (i.e. using manual positioning) using the motor.

The fine positioning procedure may be performed based on images of an imaging system, which is part of the laser applicator 506 and/or based on measurement values of an interaction measurement unit, which measures a mechanical interaction, such as a force, between the patient's eye and the laser applicator 506.

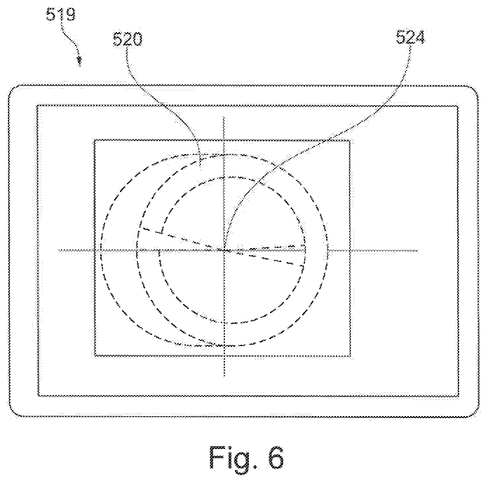

FIG. 6 schematically illustrates information, which is displayed on a display device 519 of the laser applicator 506 during the coarse positioning procedure. As can be seen from FIG. 6, the surgeon sees an image on the display device 519, which is generated by an imaging system, which is provided within the laser applicator 506. The imaging system acquires a frontal image 520 of the patient's eye using a constant object plane distance. Using the frontal image 520 of the eye 210, the surgeon can control a lateral position of the laser applicator 506 using the three-axis positioning system so that the centre of the crosshair 524 lies in the centre of the pupil of the eye 210.

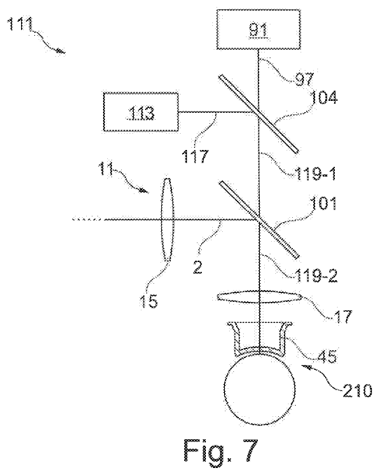

The above coarse positioning procedure may be carried out in a state in which the patient interface 45 is attached to the eye 210 (described for instance below with reference to FIG. 7). The laser applicator 506 and the patient interface 45 may be configured so that the iris and the limbus are displayed in the frontal image 520. By way of example, the portion of the frontal image 520 may be the iris of the eye 210. An image processing algorithm may include a segmentation algorithm for segmenting the frontal image. The image processing algorithm may determine one or more parameters, which depend on, or which are indicative of, a level of focus of one or more of the segmented image regions. By way of example, a segmented image region may represent the iris of the patient's eye 210.

The controller may be configured to display on the display device 519 graphical and/or textual information based on the determined parameters.

The arrangement of the imaging system within the laser applicator 506 is described in the following with reference to FIG. 7. The laser applicator 506 includes a first beam combiner 101. The first beam combiner 101 is in the beam path of the primary beam 1, which may include or consist of a treatment laser beam, between a scanning system 21 (not shown in FIG. 7) and a patient interface 45. The patient interface 45 includes a concave contact surface, which contacts the anterior surface of the cornea during the treatment. It is noted that the concave shape of the patient interface 45, which is shown in FIG. 7 is only an example and it is conceivable that the contact surface is planar or convex toward the eye 210.

The first beam combiner 101 may be in the beam path of the primary beam 1 between an upstream optical element 15 and a downstream optical element 17 of the beam delivery system 11, as it is illustrated in FIG. 7. The beam delivery system 11 is also arranged within the laser applicator 506. It is also conceivable that the laser applicator 506 includes at least a portion of a scanning system 21 for three-dimensionally scan a focus of the primary beam within the eye 210. The scanning system 21 may include an axial scanning system for scanning the laser focus along an axis of the laser beam and/or a beam deflection scanning system for scanning the laser beam through deflection of the laser beam.

Each of the optical elements 15 and 17 may include one or more optical elements, such as lenses. However, the present disclosure is not limited to such a configuration. It is also conceivable that the first beam combiner 101 is either in the primary beam path 2 of the primary beam 1 between the scanning system 21 and the beam delivery system 11 or in the beam path of the primary beam between the beam delivery system 11 and the patient interface 45.

The first beam combiner 101 may include a semi-transparent mirror and/or a prism. The semi-transparent mirror may be a dichroic mirror and/or the prism may be dichroic prism. As it is schematically illustrated in FIG. 7, the first beam combiner 101 may be configured to combine the beam path of the laser beam on the one hand with a first combined beam path 119-1, wherein the first combined beam path 119-1 includes a measurement beam path 97 of an optical coherence tomography system 91, which also may be referred to as OCT beam path, and an imaging beam path 117 of the optical imaging system 113, on the other hand. The thus obtained combined beam path may be referred to as a second combined beam path 119-2. The optical coherence tomography system 91 and the optical imaging system 113 may both relate to an optical measuring system 111. The optical imaging system 113 may have a two-dimensional light sensitive imaging sensor. The light sensitive image sensor 115 may have a two-dimensional array of light sensitive pixels. The optical coherence imaging system 91 may be configured to acquire cross-sectional images of the cornea and/or the natural lens of the eye 210. The optical imaging system 113, which has the imaging sensor may be configured to acquire the two-dimensional frontal image of the eye 210.

In the eye treatment system 500 according to the exemplary embodiment, the measurement beam path 97 of the optical coherence system 91 and the imaging beam path 117 of the optical imaging system 113 are combined using a second beam combiner, which may be a dichroic mirror 104, which is outside the beam path of the primary beam. The second beam combiner may additionally or alternatively include a prism, such as a dichroic prism.

The cross-sectional images of the OCT system 91 can be used during the fine positioning procedure for observing the intended positioning of the cornea relative to the patient interface 45.

FIG. 8 is a cross-sectional view of the parallel linkage, which includes the second arm segment 511 in the laser system according to the exemplary embodiment, which is shown in FIG. 1. The second arm segment 511 comprises two bars 552 and 553 of a four-bar linkage linking the bars 549 and 550, which is configured as a parallel linkage of the bars 552 and 553 of the second arm segment 511, which are oriented parallel relative to each other and four joints 548, which are individually labelled as 548-1, 548-2, 548-3 and 548-4, each of which having a horizontal rotation axis.

The parallel linkage also includes a counterbalancing mechanism for providing a counterbalancing that at least partially counteracts a gravitational force G acting on the laser applicator 506 (not shown in FIG. 8). In the exemplary embodiment, the counterbalancing mechanism includes a compression spring 554, which is arranged within the first bar 553 and which exerts a pulling force on a toothed belt 555, which extends into the second bar 552 via two toothed belt wheels 556, 557. Within the second bar 552, an end of the toothed belt 555 is fastened using a fastening member.

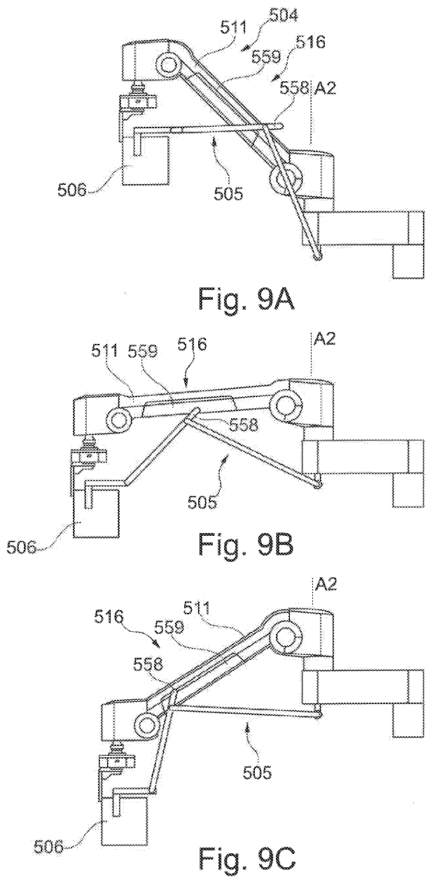

Returning to FIG. 1, the laser system according to the exemplary embodiment includes a coupling mechanism for coupling the articulated beam guide tube 505 to the second arm segment 511 of the support arm 504. This coupling mechanism 516 is illustrated in more detail in the side views of FIGS. 9A to 9C.

As can be seen from FIGS. 9A to 9C, the coupling mechanism 516 includes a first coupling member 558, which is rigidly attached or integrally connected to the articulated beam guide tube 505. Furthermore, the coupling mechanism includes a second coupling member 559, which is a corresponding coupling member to the first coupling member 558 and which is attached or integrally connected to the second arm segment 511. In the exemplary embodiment, the first and second coupling members 558, 559 are configured as a lateral guide which limits a lateral movement of the first coupling member 559 relative to a direction, which is parallel to a longitudinal axis of the second arm segment 511. In the depicted embodiment, it is ensured that during operation of the laser system 500, the articulated beam guide tube 505 does not collide with the support arm 504. Such a collision can damage the articulated beam guide tube 505 and/or the support arm 504 or can block the positioning of the laser applicator 506 using the support arm 504.

Specifically, FIGS. 9A to 9C schematically illustrate for each of three different configurations of the support arm 504 how the first and second coupling members 558, 559 are positioned relative to each other. For different inclinations of the second arm segment 511, the joint between the first and second tube segment 513 and 514 of the articulated beam guide tube 505 remains coupled to the second arm segment 511 of the support arm 504. Further, since the articulated beam guide tube extends between the laser applicator 506 and a location, where the primary beam 1 exits from the first arm segment 508 of the support arm 504, the articulated beam guide tube 505 can remain engaged with the linear guide even if the second arm segment 511 is rotated about the vertical axis A2. However, it is noted that if the lateral guide has a sufficient play, the articulated beam guide tube 505 can also be coupled to the second arm segment 511 even if the articulated beam guide tube extends from a location, where the primary beam 1 exits from the base portion 503. Additionally, or alternatively, it is also conceivable that the coupling arrangement includes a tensile force transmitting connection.

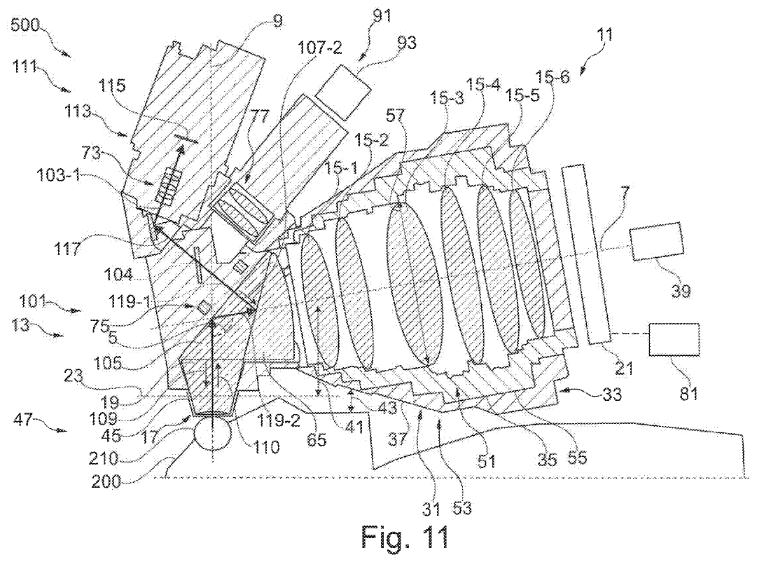

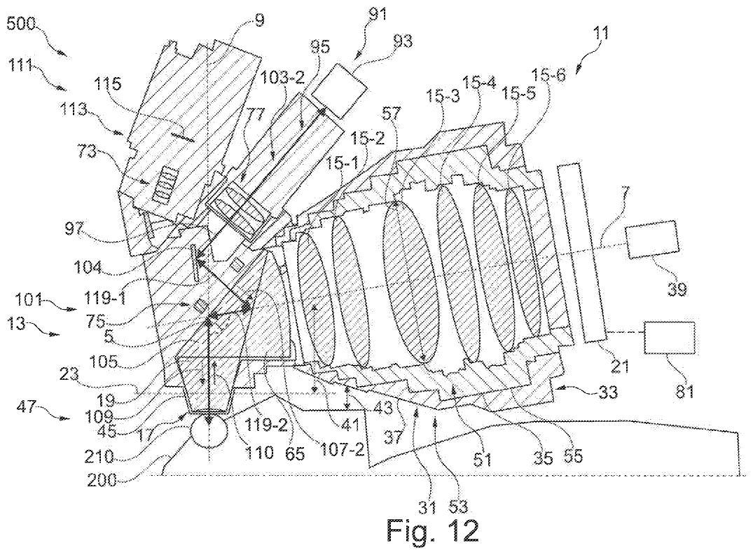

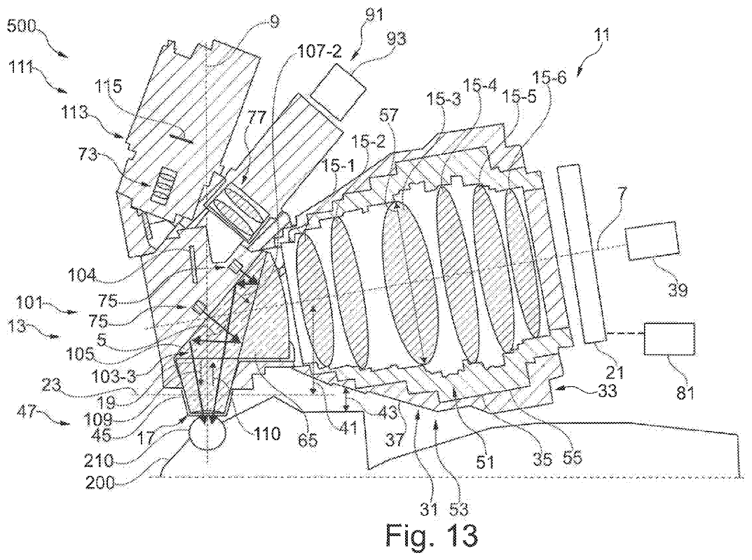

FIGS. 10 through 13 illustrate a schematic view of an ophthalmic laser system 500 configured to perform laser treatments on an eye 210 of a patient 200 according to an embodiment of the present invention. FIGS. 10 through 13 generally describe the same configuration and separately show the beam paths provided within the ophthalmic laser system 500 for different devices. FIG. 10 generally shows the primary beam paths 2 of the primary beam 1, e.g. the treatment laser beam. FIG. 11 generally shows the beam path of an optical imaging system 113. FIG. 12 generally shows the beam paths of the OCT system 91. FIG. 13 generally shows the beam paths of an illumination device 75. It is clear to the skilled person that the respective beams may be provided separately or concurrently during the operation of the ophthalmic laser system 500, as desired. It will be appreciated that the notations of the beams described herein (e.g. “primary” beam including the treatment laser beam, “secondary” beam including the OCT beam) are merely exemplarily and could of course also be used interchangeably.

As highlighted in FIG. 10, the ophthalmic laser system 500 comprises a laser beam source 39 emitting a primary beam 1 which may include or consist of a treatment laser beam, towards a beam delivery system 11 for delivering and focusing the primary beam 1 so that the primary beam 1 is guided toward the eye 210 to be treated. In different embodiments, the primary beam 1 may also include different beams e.g. one or more OCT beams, imaging beams etc. Any description within this specification with respect to the primary beam 1 accordingly applies to a treatment laser beam and may likewise apply to further beams included in the primary beam 1. In the depicted embodiment, the primary beam 1 travels towards a scanning system 21 along a first optical axis 7 defined by upstream optical elements 15 of the beam delivery system 11. It will be appreciated that the depicted beam paths and corresponding elements of the ophthalmic laser system 500 described with respect to the figures are merely exemplarily and could be well provided differently in any other suitable configuration. It will be understood that one or more further beam guiding elements such as mirrors or lenses may be provided between the laser beam source 39 and the scanning system 21, which have been omitted in the presented figure for the purpose of representability. The scanning system 21 is suitably adapted for scanning a focus of the primary beam 1 within the eye 210 or on an anterior surface of the eye 210 in one or more dimensions. The ophthalmic laser system 500 comprises a controller 81. The controller 81 is operatively coupled with the beam delivery system 11 and configured to control the scanning system 21 to position a focus of the primary beam 1 at different locations within the eye 210 and/or on an anterior surface of the cornea of the eye 210. The scanning system 21 is arranged in the beam path of the primary beam 1 upstream of the upstream optical elements 15. The scanning system 21 comprises an axial scanning system for three-dimensional positioning of a focus of the primary beam 1 in a treatment volume of the eye 210 of the patient 200.

The beam delivery system 11 further comprises a stationary deflection element 13 adapted to deflect the primary beam 1 at a deflection angle 5 toward the eye 210. Upstream of the stationary deflection element 13, a plurality of upstream optical elements 15 are arranged in a beam path of the primary beam 1. Even though in the depicted embodiment six upstream optical elements 15-1, 15-2, 15-3, 15-4, 15-5 and 15-6 are indicated, the skilled person appreciates that also more or less upstream optical elements may be provided, such as one, two three, four, five, seven or more optical elements. Thus, the primary beam 1 travels from the scanning system 21 via the upstream optical elements 15 towards the stationary deflection element 13. In the depicted embodiment, the upstream optical elements 15 are six convex shaped lenses, 15-1 to 15-6, which are transparent for the primary beam 1. It will be understood that the depicted configuration is merely schematically, and the upstream optical elements 15 may comprise other elements, such as mirrors etc. For instance, the lenses may also be shaped in a completely different manner, for instance at least partially concave, flat or of any further desired shape. In the depicted embodiment, a respective diameter 57 of each upstream optical element 15-1, 15-2 and 15-3 arranged closest to the stationary deflection element 13 along the first optical axis 7 continuously decreases toward the deflection element 13.

The upstream optical elements 15 are held by a lens mount 51 at a lens mount portion 53 of the beam delivery system 11. The lens mount 51 comprises inner portions, which are suitably adapted to an outer shape of each upstream optical element 15 to receive and hold the respective upstream optical element 15. The beam delivery system 11 further comprises a housing 31 accommodating at least partially the lens mount 51. The housing comprises an inclined portion 33, which faces the patient 200. In some embodiments, the inclined portion 33 of the housing 31 is located in contact or at a distance in a range of 0.5 mm to 20 mm to the lens mount 51. In some embodiments, additionally or alternatively, at least a portion of the inclined portion 33 of the housing 31 is formed by at least a portion of the lens mount 51. As depicted, an outer shape of the lens mount 51 and the housing 31 substantially tapers towards the stationary deflection element 13.

The stationary deflection element 13 is adapted to deflect the primary beam 1 at a deflection angle 5 toward the eye 210. As depicted, the deflection angle 5 is defined as an angle between an incident portion of the primary beam 1 along a first optical axis 7 of the upstream optical elements 15 and a deflected portion of the primary beam 1 along a second optical axis 9 of one or more downstream optical elements 17 of the beam delivery system 11. The downstream optical elements 17 are arranged in the beam path of the primary beam 1 downstream of the stationary deflection element 13.

Furthermore, the deflection angle 5 is in the depicted embodiment larger than 90°. Accordingly, the deflection angle 5 provides an increased distance or an increased free space 43 between the patient 200 and one or more of the plurality of components of the ophthalmic laser system 500 compared to a 90° deflection angle. In the depicted embodiment, the primary beam 1 is deflected so that downstream of the stationary deflection element 13, the primary beam 1 travels along the second optical axis 9, which is substantially parallel to an axis of the eye 210. The primary beam 1 accordingly travels from a point of reflection at the stationary deflection element 13 along a deflected direction 3 towards the eye 210.

The beam delivery system 11 also comprises a patient interface portion 47 with a patient interface 45 for docking the eye 210 to the ophthalmic laser system 500. As depicted, an increased distance or the increased free space 43 is provided upstream of the patient interface portion 47. Likewise, an increased distance or the increased free space 43 is provided between the patient 200 and the lens mount 51 and/or the inclined portion 33 of the housing 31. In other words, as shown in the depicted embodiment, assuming a plane 23 parallel to the general extension direction of the patient, and further assuming a distance 41 between said plane 23 and the first optical axis 7, said distance 41 can be increased compared to a 90° deflection angle. An additional free space is created by the inclined portion 33, e.g. for allowing a user to perform surgical operations or actions. Further, protruding parts of the body of the patient 200, e.g. parts of the patient's face, may be arranged in the additional space created during treatment of the patient's eye 210. In some embodiments, the exterior housing surface 37 is formed such that a clearance between the patient 200 and the housing 31 is maximized.

The exterior housing surface 37 may follow at least partially a shape of the portion of the patient 200. It is noted that the depicted shape of the patient 200 is purely schematical and for illustrative purpose only and does not indicate real proportions of body parts of a patient 200 with respect to the ophthalmic laser system 500.

As depicted, a portion of an exterior housing surface 37 of the inclined portion 33 of the housing 31 faces a portion of the patient 200. The inclined portion 33 of the housing 31 is located at a distance in close proximity to the lens mount 51. In some embodiments, a portion of the inclined portion 33 of the housing 31 may be formed by at least a portion of the lens mount 51.

An interior housing surface 35 at the inclined portion 33 of the housing 31 faces toward the lens mount 51, such that the interior housing surface 35 follows an exterior lens mount surface portion 55 of the lens mount 51. For instance, at each location of the exterior lens mount surface portion 55, the inclined portion 33 of the housing 31 may have a thickness which is in a range of 0.5 mm to 7 mm.

The ophthalmic laser system 500 comprises an optical measuring system 111 for acquiring measurement data of at least a portion of the eye 210. At least one beam of the optical measuring system 111 is combined with the primary beam 1 by the stationary deflection element 13. In the embodiment depicted in FIGS. 10 to 13, the optical measuring system 111 comprises an optical coherence tomography-OCT-system 91 configured for acquiring a cross-sectional image of at least a portion of the eye 210. The OCT system 91 comprises an OCT objective 77 having one or more optical elements, such as lenses for focusing one or more OCT beams, also referred to as second secondary beams 103-2, towards to and away from an OCT scanner 93. The beam paths of the OCT system 91 in the ophthalmic laser system 500 are highlighted in particular in FIG. 12. The OCT system 91 includes the OCT beam scanner 93 for guiding the OCT beam 95 along an OCT beam path 97 and scanning an OCT beam 95 along one or more dimensions across the eye 210. In the depicted embodiment, the OCT beam scanner 93 is separate from the scanning system 21 of the primary beam 1. However, in other embodiments, the OCT beam scanner 93 may also be integrated in the scanning system 21 of the primary beam 1. In the embodiments depicted in FIGS. 10 to 13, the optical measuring system 111 also comprises the optical imaging system 113 including a camera for acquiring a frontal image of at least a portion of the eye 210. The camera may comprise a camera objective 73 including one or more focusing optical elements, e.g. lenses to focus an imaging beam towards an image sensor 115. In other embodiments, the optical measuring system 111 may comprise additionally or alternatively a confocal microscope.

As depicted, the stationary deflection element 13 is part of a first beam combiner 101 configured to combine a beam path of the primary beam 1 and a secondary beam path of at least one secondary beam 103 and the OCT beam scanner 93 is arranged upstream the first beam combiner 101. The secondary beam path comprises one or more paths of beams of the optical measuring system 111.

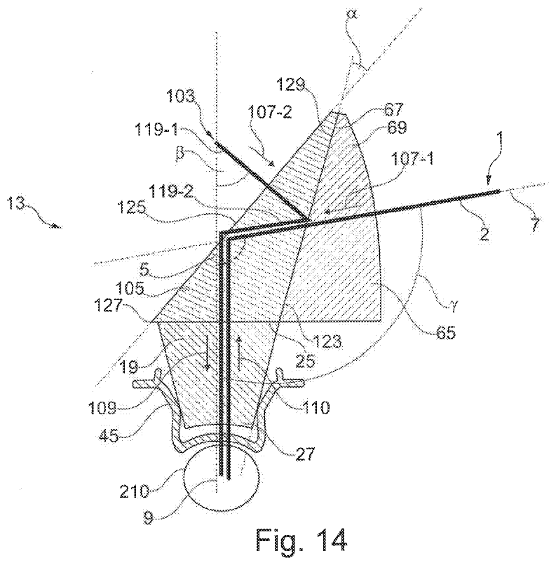

In FIG. 14, an enlarged cross-sectional view of the stationary deflection element 13 and its components, as employed for instance in the embodiments shown in FIGS. 10 to 13 is depicted. As is particularly highlighted with respect to FIG. 14, the stationary deflection element 13 comprises a reflection prism 105 for deflecting the primary beam 1. In the depicted embodiment the reflection prism 105 is a beam combining prism configured to combine a beam path of the primary beam 1 and at least one secondary beam path of at least one secondary beam 103 of the optical measuring system 111. An incidence direction 107 for each beam is depicted in the figure. In particular, the reflection prism 105 is further configured for deflecting the secondary beam 103 via one or more internal reflections from an incidence direction 107-2 towards an output direction 109. The reflection prism 105 is adapted to reflect the secondary beam 103 internally twice from a second incidence direction 107-2 towards the output direction 109. However, also more or less reflections may be conceivable dependent on the configuration of the reflection prism 105 and the desired incident and output directions. For instance, the reflection prism 105 may be a Bauernfeind prism. The reflection prism 105 has a first prism side 123 facing the primary beam 1 coming from a first incidence direction 107-1, a second prism side 125 facing one or more secondary beams 103 coming from the second incidence direction 107-2, and the third prism side 127 facing one or more reflected beams of the one or more secondary beams 103 reflected or back scattered by the eye 210 along a reflected or back scattered direction 110. The second incidence direction 107-2 is essentially perpendicular to the second prism side 125.

In the depicted embodiment, the first prism side 123 includes a reflective surface comprising at least partial optical coatings for reflecting the secondary beam 103. However, in different embodiments, additionally or alternatively also the second prism side 123 and/or the third prism side 127 may include respective reflective surfaces comprising at least partial optical coatings for reflecting the primary beam 1 and/or the secondary beam 103. One or more of the optical coatings include dichroic, notch and/or partial coatings. In the depicted embodiment, the first prism side 123 comprises a dichroic mirroring surface 129 covering at least partially the first prism side 123, wherein the dichroic mirroring surface 129 is partially transparent to the primary beam 1 and partially reflective to the secondary beam 103 having one or more different optical properties than the primary beam 1. However, it will be appreciated that in other embodiments, also no optical coatings may be provided, and a proper reflection may be for instance achieved by suitably choosing the optical properties of the materials employed.

In the depicted embodiment, the primary beam 1 and the secondary beam 103 are reflected via a total internal reflection at the second prism side 125 towards the eye 210. Similarly, when the beams are reflected or back scattered in the reflected or backscattered direction 110, they are deflected by a total internal deflection at the second prism side 125. For the skilled person, it is clear that this particular configuration is only one example of how the invention may be carried out and also different configurations are included, dependent on the shape of the reflection prism, the nature of the beams, e.g. it's wavelengths, the beam incident and exit direction and/or the provision of reflective coatings on the first, second and/or third prism side and the one or more prism materials. That is, in a different configuration, it may be well provided that in addition or alternatively the second prism side 125 and/or the third prism side 127 are adapted for enabling a total internal reflection of the primary beam 1 and/or the secondary beam 103.

The reflection prism 105 is configured to couple the primary beam 1 into the reflection prism 105 at the first prism side 123 such that the primary beam 1 travels through the reflection prism 105 towards the second prism side 125 and is then internally reflected at the second prism side 125 towards the third prism side 127. The primary beam 1 may be coupled into the reflection prism 105 at the first prism side 123 essentially orthogonally at an angle in a range of about 82° to 92°. However, in different configuration, the primary beam may also be coupled into the reflection prism 105 at the first prism side 123 at a more inclined angle, such as at an angle in a range of 85° to 95° or even in a range of 80° to 100°. The primary beam 1 is internally reflected at the second prism side 125 of about 50°. However, in different configuration, the primary beam may also be internally reflected at the second prism side 125 at an angle in a range of 45° to 85°. The primary beam 1 is then coupled out of the reflection prism 105 at the third prism side 127 towards the eye 210. Furthermore, the primary beam 1 is coupled out of the reflection prism 105 essentially perpendicular to the third prism side 127 along the output direction 109.

Likewise, the reflection prism 105 is configured to couple the secondary beam 103 into the reflection prism 105 at the second prism side 125 such that the secondary beam 103 travels through the reflection prism 105 towards the first prism side 123 and is reflected at the dichroic mirroring surface 129 of the first prism side 123 towards the second prism side 125. The secondary beam 103 then is internally reflected at the second prism side 125 towards the third prism side 127 and coupled out of the reflection prism 105 at the third prism side 127 towards the eye 210. The secondary beam 103 and/or the reflected or back scattered secondary beam 103 may be reflected at the dichroic mirroring surface 129 of the first prism side 123 at an angle in a range of 45° to 85°, preferably of about 50°. Also the secondary beam 103 and/or the reflected or back scattered secondary beam 103 may be internally reflected at the second prism side 125 at an angle in a range of 45° to 85°, preferably of about 50°.

The outcoupled secondary beam 103 is then at least partially reflected or back scattered by one or more features of the eye 210 along a reflected or back scattered direction 110 being essentially opposite to the output direction 109. The reflection prism 105 is configured to couple the reflected or back scattered secondary beam 103 into the reflection prism 105 at the third prism side 127 such that the reflected or back scattered secondary beam 103 travels through the reflection prism 105 towards the second prism side 125 and is then internally reflected at the second prism side 125 towards the first prism side 123. The reflected or back scattered secondary beam 103 is then reflected at the dichroic mirroring surface 129 of the first prism side 123 towards the second prism side 125, and the reflected or back scattered secondary beam 103 is then coupled out of the reflection prism 105 at the second prism side 125 towards one or more detectors adapted to detect the reflected or back scattered secondary beam 103. In the depicted embodiment, the reflected or back scattered secondary beam 103 is coupled out of the reflection prism 105 essentially perpendicular to the second prism side 125. However, in a different configuration, the beam may also be coupled out in an inclined manner. As shown with respect to FIGS. 10 to 13, the stationary deflection element 13 is part of the first beam combiner 101. The stationary deflection element 13 is formed by the reflection prism 105 and the reflection prism 105 is a beam combining prism configured to combine the primary beam 1 and the secondary beam 103 to a common beam path along the output direction 109. Further, the reflection prism 105 is configured to combine a beam path of the primary beam 1 and at least one secondary beam path of at least one secondary beam 103 of the optical measuring system 111 for acquiring measurement data of at least a portion of the eye 210 or for illuminating at least a portion of the eye 210.

Referring in particular to FIGS. 11 and 12, upstream of the first beam combiner 101 a second beam combiner including a dichroic mirror 104 is arranged in a beam path of the secondary beam 103 upstream of the second prism side 125. The dichroic mirror 104 is at least partially transparent to a first secondary beam 103-1 (cf. FIG. 11) and at least partially reflective to a second secondary beam 103-2 (cf. FIG. 12) having one or more different optical properties than the first secondary beam 103-1. Hence, the dichroic mirror 104 is adapted to combine the first secondary beam 103-1 and the second secondary beam 103-2 along the second incidence direction 107-2.

The downstream optical elements 17 comprise a truncated cone-shaped optical element 19 arranged between the stationary deflection element 13 and the eye 210 for guiding a primary beam 1 of the ophthalmic laser system 500 toward the eye 210. The truncated cone-shaped optical element 19 tapers along the second optical axis 9 towards the eye 210. In the depicted embodiments of FIGS. 10 to 14, the truncated cone-shaped optical element 19 is part of the first beam combiner 101. As highlighted in FIG. 14, the truncated cone-shaped optical element 19 comprises an essentially flat first cone end surface 25 facing the third prism side 127 and an oppositely arranged second cone end surface 27 facing the eye 210. The essentially flat first cone end surface 25 contacts at least partially the third prism side 127. The patient interface 45 is arranged between the second cone end surface 27 and the eye 210 for docking the eye 210 to the ophthalmic laser system 500. The truncated cone-shaped optical element 19 is arranged between the eye 210 and the stationary deflection element 13 adapted to deflect the primary beam 1 at a deflection angle 5 toward the eye 210. The truncated cone-shaped optical element provides the primary beam 1 to the eye 210 at a numerical aperture of 0.1 to 0.4, preferably of 0.18 to 0.25, more preferably of about 0.2. The stationary deflection element 13 is adapted to combine one or more secondary beams 103 of the optical measuring system 111 with the primary beam 1 and the truncated cone-shaped optical element 19 is adapted to guide the combined primary beam 1 and the one or more secondary beams 103 towards the eye 210.

FIG. 13 particularly shows beam paths of the illumination device 75 configured for illuminating at least a portion of the eye 210. The illumination device 75 includes two light sources adapted to emit one or more secondary beams 103-3 towards a reflection prism 105, wherein the secondary beams 103-3 comprise illumination light beams. However, also only one light source or more than two light sources may be provided, in different embodiments, as desired. In the depicted embodiment, the illumination device 75 comprises an LED light source, which is formed as a ring extending in a plane essentially parallel to a surface of the reflection prism 105 upon which the illumination light beams are irradiated. However, of course also different forms of light sources and respective arrangements may be provided. Hence, the respective secondary beams 103-3 are irradiated essentially perpendicular to a second prism surface 125 along the second incidence direction 107-2 (cf. also FIG. 14). The reflection prism 105 is adapted to deflect a beam path of the secondary beam 103 from the second incidence direction 107-2 towards the output direction 109. This may occur in a range of about 45° to 85°, preferably of about 50° dependent on the configurations of the prism and the second incident direction 107-2 or output directions 109. The secondary beam 103 is deflected under a secondary beam deflection angle β, wherein the secondary beam deflection angle β is defined between the second incidence direction 107-2 and the output direction 109. The first prism side 123 and the second prism side 125 contact each other at a functional prism angle α, wherein the functional prism angle α is about 25°. However, in different embodiments the prism may comprise different prism angles α, such as in a range of 25.2° to 42.5°. The reflection prism 105 guides the secondary beam 103 along a first combined beam path 119-1.

The reflection prism 105 guides the primary beam 1 along a primary beam path 2. As depicted, the primary beam 1 is deflected under a primary beam deflection angle γ (also referred to as 5 in the figures), wherein the primary beam deflection angle γ is defined between the first incidence direction 107-1 and the output direction 109. The functional prism angle α, the secondary beam deflection angle β and the primary beam deflection angle γ may follow the relationships: β=2*α and γ=2*β. The primary beam 1 and the secondary beam 103 are accordingly combined and guided along a second combined beam path 119-2.

The ophthalmic laser system 500 also includes an upstream optical coupling element 65 arranged between the stationary deflection element 13 and the upstream optical elements 15 along the first optical axis 7. However, in different embodiments, also more than one upstream optical coupling element 65 may be provided. The upstream optical coupling element 65 is also part of the first beam combiner 101. In the depicted embodiment, the upstream optical coupling element 65 is an input prism lens. The upstream optical coupling element 65 comprises an essentially flat first upstream optical coupling element surface 67 facing the first prism side 123 and an oppositely essentially convexly curved second upstream optical coupling element surface 69 facing the upstream optical elements 15. As shown, the essentially flat first upstream optical coupling element surface 67 contacts the first prism side 123. In different embodiments, only a partial contact may be provided between the flat first upstream optical coupling element surface 67 and the first prism side 123.

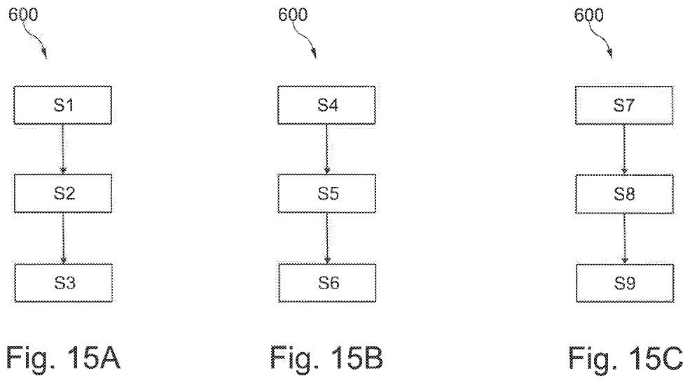

FIG. 15 shows flowcharts of embodiments of methods 600 for operating an ophthalmic laser system 500 according to the present invention.

As depicted in FIG. 15A, in the method 600 a step S1 is provided, which includes providing a beam delivery system 11 for focusing the primary beam 1. The primary beam 1 may be focused by the beam delivery system 11 so that the primary beam 1 travels toward the eye 210 to be treated. Further, a step S2 is provided, which includes providing a scanning system 21 for scanning a focus of the primary beam 1 within the eye 210 or on an anterior surface of the eye 210 in two or more dimensions. As described above, the beam delivery system 11 comprises a stationary deflection element 13 adapted to deflect the primary beam 1 at a deflection angle 5 toward the eye 210. The deflection angle 5 is defined as an angle between an incident portion of the primary beam 1 along a first optical axis 7 of one or more upstream optical elements 15 of the beam delivery system 11, wherein the upstream optical elements 15 are arranged in a beam path of the primary beam 1 upstream of the stationary deflection element 13 and a deflected portion of the primary beam 1 along a second optical axis 9 of one or more downstream optical elements 17 of the beam delivery system 11. The downstream optical elements 17 are arranged in the beam path of the primary beam 1 downstream of the stationary deflection element 13. Further, a step S3 is provided, which includes deflecting the primary beam 1 at angle 5 larger than 90°.

As depicted in FIG. 15B, in the method 600 a step S4 is provided, which includes providing a beam delivery system 11 for focusing the primary beam 1 so that the primary beam 1 travels toward an eye 210 to be treated. Further, a step S5 is provided which includes providing a reflection prism 105 adapted to internally deflect the primary beam 1 toward the eye 210. Further, a step S6 is provided, which includes irradiating the primary beam 1 toward the reflection prism 105, wherein the primary beam 1 is internally deflected from the reflection prism 105 toward the eye 210 to be treated.

As depicted in FIG. 15C, in the method 600 a step S7 is provided, which includes providing a truncated cone-shaped optical element 19. Further, a step S8 is provided, which includes arranging S8 the truncated cone-shaped optical element 19 between the eye 210 and a stationary deflection element 13 adapted to deflect the primary beam 1 at a deflection angle 5 toward the eye 210, wherein the truncated cone-shaped optical element 19 tapers along a second optical axis 9 towards the eye 210. Further, a step S9 is provided, which includes guiding the primary beam 1 of the ophthalmic laser system 500 from the deflection element 13 via the truncated cone-shaped optical element 19 to the eye 210 to be treated.

The above embodiments as described are only illustrative, and not intended to limit the technique approaches of the present invention. Although the present invention is described in detail referring to the preferable embodiments, those skilled in the art will understand that the technique approaches of the present invention can be modified or equally displaced without departing from the protective scope of the claims of the present invention. In the claims, the words “comprising” or “including” do not exclude other elements or steps, and the indefinite article “a” or “an” does not exclude a plurality. Any reference signs in the claims should not be construed as limiting the scope.

### List of reference signs

1 primary beam
2 primary beam path
3 deflected direction
5 deflection angle
7 first optical axis
9 second optical axis
11 beam delivery system
13 stationary deflection element
15 upstream optical elements
17 downstream optical elements
19 truncated cone-shaped optical element
21 scanning system
23 plane
25 first cone end surface
27 second cone end surface
31 housing
33 inclined portion
35 interior housing surface
37 exterior housing surface
39 laser beam source
41 distance to plane
43 distance patient
45 patient interface
47 patient interface portion
51 lens mount
53 lens mount portion
55 exterior lens mount surface portion
57 diameter
65 upstream optical coupling element
67 first upstream optical coupling element surface
69 second upstream optical coupling element surface
73 camera objective
75 illumination device
77 OCT objective
81 controller
91 OCT system
93 OCT scanner
95 OCT beam
97 OCT beam path
101 first beam combiner
103 secondary beam
103-1 first secondary beam
103-2 second secondary beam
103-3 third secondary beam
104 dichroic mirror
105 reflection prism
107 incidence direction
107-1 first incidence direction
107-2 second incidence direction
109 output direction
110 reflection or back scattered direction
111 optical measuring system
113 optical imaging system
115 image sensor
117 imaging beam path
119-1 first combined beam path
119-2 second combined beam path
123 first prism side
125 second prism side
127 third prism side
129 dichroic mirroring surface
200 patient
210 eye
500 ophthalmic laser system
502 housing
503 base portion
504 support arm
505 beam guide tube
506 laser applicator
508 first arm segment
509 motorized positioning system
510 operable control unit
511 second arm segment
512 intermediate joint
513 first tube segment
514 second tube segment
516 coupling mechanism
517 handle
518 control elements
519 display device
520 frontal image
521 first location
523 second location
524 crosshair
548 joints
549 bar
550 bar
552 bar
553 bar
554 compression spring
555 toothed belt
556 toothed belt wheel
557 toothed belt wheel
558 first coupling member
559 second coupling member
A1 vertical rotation axis
A2 vertical rotation axis
A3 horizontal rotation axis
A5 horizontal rotation axis
A6 vertical rotation axis
600 method
S1 to S9 method steps
α functional prism angle
β secondary beam deflection angle
γ primary beam deflection angle
   Before we go on to set out the claims, we first set out the following clauses describing some prominent features of certain embodiments of the present disclosure.
Clause 1. An ophthalmic laser system configured to perform laser treatments on an eye of a patient using a primary beam comprising a treatment laser beam, the ophthalmic laser system comprising a plurality of components including:
a beam delivery system for focusing the primary beam,
a scanning system for scanning a focus of the primary beam within the eye or on an anterior surface of the eye in two or more dimensions,
wherein the beam delivery system comprises a stationary deflection element adapted to deflect the primary beam at a deflection angle toward the eye,
wherein the deflection angle is defined as an angle between:
   an incident portion of the primary beam along a first optical axis of one or more upstream optical elements of the beam delivery system, wherein the upstream optical elements are arranged in a beam path of the primary beam upstream of the stationary deflection element; and
   a deflected portion of the primary beam along a second optical axis of one or more downstream optical elements of the beam delivery system, wherein the downstream optical elements are arranged in the beam path of the primary beam downstream of the stationary deflection element;
wherein the deflection angle is larger than 90°.
Clause 2. The ophthalmic laser system according to the preceding clause,
wherein the deflection angle provides an increased distance or an increased free space between the patient and one or more of the plurality of components of the ophthalmic laser system compared to a 90° deflection angle.
Clause 3. The ophthalmic laser system according to the preceding clause,
wherein the beam delivery system comprises a patient interface portion comprising a patient interface for docking the eye to the ophthalmic laser system,
wherein the increased distance or the increased free space is provided upstream of the patient interface portion.
Clause 4. The ophthalmic laser system according to one of the preceding clauses 2 or 3,
wherein the optical elements comprise one or more lenses, and
wherein the ophthalmic laser system further comprises a lens mount arranged at a lens mount portion of the beam delivery system upstream of the patient interface portion,
wherein the lens mount is adapted to hold one or more of the upstream optical elements,
wherein the increased distance or the increased free space is provided between the patient and the lens mount.
Clause 5. The ophthalmic laser system according to one of the preceding clauses 2 to 4,
wherein the beam delivery system comprises a housing,
wherein the housing is adapted to accommodate at least partially one or more of the plurality of components of the ophthalmic laser system,
wherein the housing comprises an inclined portion upstream of the patient interface portion, wherein the inclined portion faces towards the patient,
wherein the increased distance or the increased free space is provided between the patient and the inclined portion of the housing.
Clause 6. The ophthalmic laser system according to the preceding clause in combination with clause 4,
wherein:
the inclined portion of the housing is located in contact or at a distance in a range of 0.5 mm to 20 mm to the lens mount; preferably in a range of 1.0 mm to 15 mm, and most preferably in a range of 1.75 mm to 10,9 mm and/or
at least a portion of the inclined portion of the housing is formed by at least a portion of the lens mount.
Clause 7. The ophthalmic laser system according to one of the preceding clauses,
wherein the deflection angle is larger than 95°, preferably larger than 100°, more preferably larger than 105°.
Clause 8. The ophthalmic laser system (500) according to one of the preceding clauses,
wherein the deflection angle (5) is smaller than 180°, preferably smaller than 175°, more preferably smaller than 170°.
Clause 9. The ophthalmic laser system according to one of the preceding clauses,
wherein the primary beam comprises a single beam or a plurality of beams.
Clause 10 The ophthalmic laser system according to one of the preceding clauses,
wherein the primary beam is deflected so that downstream of the stationary deflection element, the primary beam travels along the second optical axis, which is substantially parallel to an axis of the eye.
Clause 11. The ophthalmic laser system according to the preceding clause,
wherein an interior housing surface of at least a portion of the inclined portion of the housing faces toward at least a portion of the lens mount.
Clause 12. The ophthalmic laser system according to the preceding clause,
wherein at least a portion of the interior housing surface follows an exterior lens mount surface portion of the lens mount.
Clause 13. The ophthalmic laser system according to the preceding clause,
wherein at each location of the exterior lens mount surface portion, the inclined portion of the housing has a thickness which is in a range of 0.5 mm to 7 mm.
Clause 14. The ophthalmic laser system according to one of the preceding clauses 5 to 13,
wherein at least a portion of an exterior housing surface of at least a portion of the inclined portion of the housing faces a portion of the patient, and
wherein the exterior housing surface follows at least partially a shape of the portion of the patient.
Clause 15. The ophthalmic laser system according to one of the preceding clauses 5 to 14,
wherein an additional free space is created by the inclined portion for allowing a user to perform surgical operations.
Clause 16. The ophthalmic laser system according to the preceding clause,
wherein the exterior housing surface is formed such that a clearance between the patient and the housing is maximized.
Clause 17. The ophthalmic laser system according to one of the preceding clauses,
wherein the scanning system is arranged in the beam path of the primary beam upstream of the upstream optical elements.
Clause 18. The ophthalmic laser system according to one of the preceding clauses,
wherein the scanning system comprises:
an axial scanning system for three-dimensional positioning of a focus of the primary beam in a treatment volume of the eye of the patient.
Clause 19. The ophthalmic laser system according to one of the preceding clauses,
a diameter of three or four upstream optical elements arranged closest to the stationary deflection element along the first optical axis continuously decreases toward the stationary deflection element.
Clause 20. The ophthalmic laser system according to one of the preceding clauses 4 to 19,
wherein an outer shape of the lens mount and/or the housing substantially tapers towards the stationary deflection element.
Clause 21. The ophthalmic laser system according to one of the preceding clauses,
wherein the beam delivery system is part of a displaceable laser applicator,
wherein the ophthalmic laser system comprises a support arm for displaceably supporting the laser applicator.
Clause 22. The ophthalmic laser system according to the preceding clause,
wherein the support arm or at least an arm segment of the support arm is configured to be rotatable about a horizontal or substantially horizontal rotation axis and/or a vertical or substantially vertical rotation axis.
Clause 23. The ophthalmic laser system according to the preceding clause,
wherein the support arm is movably hinged at a base portion of the ophthalmic laser system so that the laser applicator is positionable relative to the base portion in one or more, in particular three, dimensions.
Clause 24. The ophthalmic laser system according to the preceding clause,
wherein the laser applicator is positionable relative to the base portion while maintaining an essentially vertical orientation of the laser applicator.
Clause 25. The ophthalmic laser system according to the preceding clauses 23 or 24,
wherein a positioning of the laser applicator relative to the base portion includes a rotation of the laser applicator relative to the base portion in one or more, in particular three, dimensions.
Clause 26. The ophthalmic laser system according to one of the preceding clauses 21 to 25,
wherein the support arm comprises a first arm segment and a second arm segment which are connected to each other in series via an intermediate joint; wherein the first arm segment is rotatable about a vertical or substantially vertical axis and the second arm segment is rotatable about a horizontal or substantially horizontal axis.
Clause 27. The ophthalmic laser system according to the preceding clause,
wherein the intermediate joint is configured to allow the second arm segment to rotate about the horizontal or substantially horizontal axis and about a vertical or substantially vertical axis.
Clause 28. The ophthalmic laser system according to one of the preceding clauses,
wherein the ophthalmic laser system comprises a controller operatively coupled with the beam delivery system and configured to control the scanning system to position a focus of the primary beam at different locations within the eye or on an anterior surface of the cornea of the eye.
Clause 29. The ophthalmic laser system according to one of the preceding clauses,
wherein the ophthalmic laser system comprises an optical measuring system for acquiring measurement data of at least a portion of the eye,
wherein at least one beam of the optical measuring system is combined with the primary beam by the stationary deflection element.
Clause 30. The ophthalmic laser system according to one of the preceding clauses 1 to 28,
wherein the ophthalmic laser system comprises an optical measuring system for acquiring measurement data of at least a portion of the eye,
wherein at least one beam of the optical measuring system is combined with the primary beam upstream of the stationary deflection element.
Clause 31. The ophthalmic laser system according to one of the preceding clauses 29 or 30,
wherein the optical measuring system comprises an optical coherence tomography system configured for acquiring a cross-sectional image of at least a portion of the eye.
Clause 32. The ophthalmic laser system according to the preceding clause,
wherein the OCT system includes an OCT beam scanner for scanning an OCT beam along one or more dimensions across the eye.
Clause 33. The ophthalmic laser system according to the preceding clause,
wherein the OCT beam scanner is separate from the scanning system of the primary beam.
Clause 34. The ophthalmic laser system according to clause 32,
wherein the OCT beam scanner is integrated in the scanning system of the primary beam.
Clause 35. The ophthalmic laser system according to one of the preceding clauses 29 to 34,
wherein the optical measuring system comprises an optical imaging system for acquiring a frontal image of at least a portion of the eye.
Clause 36. The ophthalmic laser system according to one of the preceding clauses 29 to 35,
wherein the optical measuring system further comprises a camera.
Clause 37. The ophthalmic laser system according to one of the preceding clauses 29 to 36,
wherein the optical measuring system further comprises a confocal microscope.
Clause 38. The ophthalmic laser system according to one of the preceding clauses,
wherein the stationary deflection element is part of a first beam combiner configured to combine a beam path of the primary beam and at least one secondary beam path of at least one secondary beam.
Clause 39. The ophthalmic laser system according to the preceding clause,
wherein one or more, preferably all, elements of the first beam combiner are made of an optical material with refractive index above 1.7, more preferably in a range of 1.77 to 1.84.
Clause 40. The ophthalmic laser system according to one of the preceding clauses 38 or 39 in combination with one of clauses 31 to 34,
wherein the OCT beam scanner is arranged upstream the first beam combiner.
Clause 41. The ophthalmic laser system according to one of the preceding clauses 38 to 40,
wherein the secondary beam path comprises one or more beam paths of beams of the optical measuring system.
Clause 42. The ophthalmic laser system according to one of the preceding clauses 38 to 41,
wherein the stationary deflection element comprises a reflection prism for deflecting the primary beam.
Clause 43. The ophthalmic laser system according to the preceding clause,
wherein the reflection prism is further configured for deflecting the secondary beam via one or more internal reflections from a second incidence direction towards an output direction.
Clause 44. The ophthalmic laser system of one of the preceding clauses 38 to 43,
wherein the stationary deflection element is part of the first beam combiner.
Clause 45. The ophthalmic laser system of the preceding clause,
wherein the stationary deflection element is formed by the reflection prism, wherein the reflection prism is a beam combining prism configured to combine the primary beam and the secondary beam to a common beam path along the output direction.
Clause 46. The ophthalmic laser system according to one of the preceding clauses 43 to 45,
wherein the reflection prism is adapted to reflect the secondary beam internally twice from the second incidence direction towards the output direction.
Clause 47. The ophthalmic laser system according to one of the preceding clauses,
further comprising an illumination device configured for illuminating at least a portion of the eye.
Clause 48. The ophthalmic laser system according to the preceding clause,
wherein the illumination device includes one or more light sources adapted to emit one or more secondary beams towards a reflection prism, wherein the one or more secondary beams comprise illumination light beams.
Clause 49. The ophthalmic laser system according to one of the preceding clauses 47 or 48,
wherein the illumination device comprises at least one LED light source.
Clause 50. The ophthalmic laser system according to one of the preceding clauses 48 or 49,
wherein the LED light source is formed as a ring extending in a plane essentially parallel to a surface of the reflection prism upon which the illumination light beams are irradiated.
Clause 51. The ophthalmic laser system according to one of the preceding clauses 42 to 50,
wherein the reflection prism is adapted to deflect a beam path of the secondary beam from the second incidence direction towards the output direction in a range of about 45° to 85°, preferably of about 50°.
Clause 52. The ophthalmic laser system according to one of the preceding clauses 42 to 51,
wherein the reflection prism is a Bauernfeind prism.
Clause 53. The ophthalmic laser system according to one of the preceding clauses 42 to 52,
wherein the reflection prism has a first prism side facing the primary beam coming from a first incidence direction, a second prism side facing one or more secondary beams coming from the second incidence direction, and a third prism side facing one or more reflected beams of the one or more secondary beams reflected or back scattered by the eye along a reflected or back scattered direction.
Clause 54. The ophthalmic laser system according to the preceding clause,
wherein one or more of the first prism side, the second prism side and the third prism side include reflective surfaces comprising at least partial optical coatings for reflecting the primary beam and/or the secondary beam.
Clause 55. The ophthalmic laser system according to the preceding clause,
wherein one or more of the first prism side, the second prism side and the third prism side are additionally or alternatively adapted to for enabling a total internal reflection the primary beam and/or the secondary beam.
Clause 56. The ophthalmic laser system according to the preceding clause,
wherein one or more of the optical coatings include dichroic, notch and/or partial coatings.
Clause 57. The ophthalmic laser system according to one of the preceding clauses 53 to 56,
wherein the first prism side and the second prism side contact each other at a functional prism angle, wherein the functional prism angle is in the range of 22.5° to 42.5°, preferably of about 25°.
Clause 58. The ophthalmic laser system according to one of the preceding clauses 53 to 57,
wherein the reflection prism is adapted to guide the secondary beam along a combined beam path,
wherein the secondary beam is deflected under a secondary beam deflection angle, wherein the secondary beam deflection angle is defined between the second incidence direction and the output direction, wherein the secondary beam deflection angle is in a range of 45° to 85°, preferably of about 50°.
Clause 59. The ophthalmic laser system according to one of the preceding clauses 53 to 58,
wherein the reflection prism is adapted to guide the primary beam along a primary beam path,
wherein the primary beam is deflected under a primary beam deflection angle, wherein the primary beam deflection angle is defined between the first incidence direction and the output direction.
Clause 60. The ophthalmic laser system according to the preceding clause in combination with clauses 57 and 58,
wherein the functional prism angle, the secondary beam deflection angle and the primary beam deflection angle follow the relationships: β=2*α and γ=2*β.
Clause 61. The ophthalmic laser system according to one of the preceding clauses 53 to 60,
wherein the second incidence direction is essentially perpendicular to the second prism side.
Clause 62. The ophthalmic laser system according to the preceding clauses 53 to 61,
wherein the reflected or back scattered direction is essentially perpendicular to the third prism side or wherein the reflected or back scattered direction is inclined at an angle of up to 45° with respect to a direction perpendicular to the third prism side.
Clause 63. The ophthalmic laser system according to one of the preceding clauses 53 to 62,
wherein the reflection prism is configured to couple the primary beam into the reflection prism at the first prism side such that the primary beam travels through the reflection prism towards the second prism side and is then internally reflected at the second prism side towards the third prism side, and
wherein the primary beam is then coupled out of the reflection prism at the third prism side towards the eye.
Clause 64. The ophthalmic laser system according to the preceding clause,
wherein the primary beam is coupled out of the reflection prism essentially perpendicular to the third prism side along the output direction.
Clause 65. The ophthalmic laser system according to one of the preceding clauses 53 to 64,
wherein the first prism side comprises a dichroic mirroring surface covering at least partially the first prism side,
wherein the dichroic mirroring surface is at least partially transparent to the primary beam and at least partially reflective to the secondary beam having one or more different optical properties than the primary beam.
Clause 66. The ophthalmic laser system according to one of the preceding clauses 53 to 65,
wherein the reflection prism is configured to couple the secondary beam into the reflection prism at the second prism side such that the secondary beam travels through the reflection prism towards the first prism side and is reflected at the dichroic mirroring surface of the first prism side towards the second prism side,
wherein the secondary beam then is internally reflected at the second prism side towards the third prism side, and
wherein the secondary beam is coupled out of the reflection prism at the third prism side towards the eye.
Clause 67. The ophthalmic laser system according to the preceding clause,
wherein the outcoupled secondary beam is then at least partially reflected or back scattered by one or more features of the eye along a reflected or back scattered direction being essentially opposite to the output direction,
wherein the reflection prism is configured to couple the reflected or back scattered secondary beam into the reflection prism at the third prism side such that the reflected or back scattered secondary beam travels through the reflection prism towards the second prism side and is then internally reflected at the second prism side towards the first prism side,
wherein the reflected or back scattered secondary beam is then reflected at the dichroic mirroring surface of the first prism side towards the second prism side, and
wherein the reflected or back scattered secondary beam is then coupled out of the reflection prism at the second prism side towards one or more detectors adapted to detect the reflected or back scattered secondary beam.
Clause 68. The ophthalmic laser system according to the preceding clause,
wherein the reflected or back scattered secondary beam is coupled out of the reflection prism essentially perpendicular to the second prism side.
Clause 69. The ophthalmic laser system according to one of to the preceding clauses 53 to 68,
wherein the primary beam is coupled into the reflection prism at the first prism side at an angle in a range of 80° to 100°, preferably 85° to 95°, more preferably 82° to 92°.
Clause 70. The ophthalmic laser system according to one of the preceding clauses 53 to 69,
wherein the primary beam is internally reflected at the second prism side at an angle in a range of larger than 90° to 170°, preferably of about 100°.
Clause 71. The ophthalmic laser system according to one of the preceding clauses 53 to 70,
wherein the secondary beam and/or the reflected or back scattered secondary beam is reflected at the dichroic mirroring surface of the first prism side at an angle in a range of 45° to 85°, preferably of about 50°.
Clause 72. The ophthalmic laser system according to one of the preceding clauses 53 to 71,
wherein the secondary beam and/or the reflected or back scattered secondary beam is internally reflected at the second prism side at an angle in a range of larger than 90° to 170°, preferably of about 100°.
Clause 73. The ophthalmic laser system according to one of the preceding clauses 53 to 72,
wherein the first beam combiner comprises a dichroic mirror arranged in a beam path of the secondary beam upstream of the second prism side,
wherein the dichroic mirror is at least partially transparent to a first secondary beam and at least partially reflective to a second secondary beam having one or more different optical properties than the first secondary beam,
wherein the dichroic mirror is adapted to combine the first secondary beam and the second secondary beam along the second incidence direction.
Clause 74. The ophthalmic laser system of one of the preceding clauses,
wherein the downstream optical elements comprise a truncated cone-shaped optical element arranged between the stationary deflection element and the eye tapering along the second optical axis towards the eye.
Clause 75. The ophthalmic laser system of the preceding clause in combination with clause 38,
wherein the truncated cone-shaped optical element is part of the first beam combiner.
Clause 76. The ophthalmic laser system of one of the preceding clauses 53 to 75,
wherein the truncated cone-shaped optical element comprises an essentially flat first cone end surface facing the third prism side and an oppositely arranged second cone end surface facing the eye.
Clause 77. The ophthalmic laser system of the preceding clause,
wherein the essentially flat first cone end surface contacts at least partially the third prism side.
Clause 78. The ophthalmic laser system of one of the preceding clauses 76 or 77 in combination with clause 3,
wherein the patient interface is arranged between the second cone end surface and the eye.
Clause 79. The ophthalmic laser system of one of the preceding clauses 74 to 78,
wherein the truncated cone-shaped optical element is adapted to provide the primary beam to the eye at a numerical aperture of 0.1 to 0.4, preferably of 0.18 to 0.25, more preferably of about 0.2.
Clause 80. The ophthalmic laser system of one of the preceding clauses,
further comprising at least one upstream optical coupling element arranged between the stationary deflection element and the upstream optical elements along the first optical axis.
Clause 81. The ophthalmic laser system of the preceding clause in combination with clause 38,
wherein the upstream optical coupling element is part of the first beam combiner.
Clause 82. The ophthalmic laser system of one of the preceding clauses 79 to 81,
wherein the upstream optical coupling element is an input prism lens.
Clause 83. The ophthalmic laser system of one of the preceding clauses 80 to 82 in combination with clause 53,
wherein the upstream optical coupling element comprises an essentially flat first upstream optical coupling element surface facing the first prism side and an oppositely essentially convexly curved second upstream optical coupling element surface facing the upstream optical elements.
Clause 84. The ophthalmic laser system of the preceding clause,
wherein the essentially flat first upstream optical coupling element surface at least partially contacts the first prism side.
Clause 85. The ophthalmic laser system of one of the preceding clauses,
wherein the laser system is a stationary system or a mobile system.
Clause 86. The ophthalmic laser system of one of the preceding clauses,
wherein the beam delivery system is configured for focusing the primary beam so that the primary beam travels toward the eye to be treated.
Clause 87. An ophthalmic laser system configured to perform laser treatments on an eye of a patient using a primary beam comprising a treatment laser beam, the ophthalmic laser system comprising:
a beam delivery system for focusing the primary beam,
wherein the beam delivery system comprises a reflection prism adapted to internally deflect the primary beam toward the eye.
Clause 88. The ophthalmic laser system according to the preceding clause,
wherein the reflection prism is a Bauernfeind prism.
Clause 89. The ophthalmic laser system according to one of the preceding clauses 87 or 88,
wherein the reflection prism is a beam combining prism configured to combine a beam path of the primary beam and at least one secondary beam path of at least one secondary beam of an optical measuring system for acquiring measurement data of at least a portion of the eye or for illuminating at least a portion of the eye.
Clause 90. The ophthalmic laser system according to the preceding clause,
wherein the optical measuring system comprises an optical coherence tomography system configured for acquiring a cross-sectional image of at least a portion of the eye.
Clause 91. The ophthalmic laser system according to the preceding clause,
wherein the OCT system includes an OCT beam scanner for scanning an OCT beam along one or more dimensions across the eye.
Clause 92. The ophthalmic laser system according to the preceding clause,
wherein the OCT beam scanner is separate from the scanning system of the primary beam.
Clause 93. The ophthalmic laser system according to one of the preceding clauses 91 or 92,
wherein the OCT beam scanner is integrated in the scanning system of the primary beam.
Clause 94. The ophthalmic laser system according to one of the preceding clauses 87 to 93,
further comprising an illumination device configured for illuminating at least a portion of the eye.
Clause 95. The ophthalmic laser system according to the preceding clause,
wherein the illumination device includes one or more light sources adapted to emit one or more secondary beams towards the reflection prism, wherein the one or more secondary beams comprise illumination light beams.
Clause 96. The ophthalmic laser system according to the preceding clause,
wherein the illumination device comprises at least one LED light source.
Clause 97. The ophthalmic laser system according to the preceding clause,
wherein the LED light source is formed as a ring extending in a plane essentially parallel to a surface of the reflection prism upon which the illumination light beams are irradiated.
Clause 98. The ophthalmic laser system according to one of the clauses 87 to 97,
wherein the reflection prism is adapted to reflect the secondary beam internally twice from an incidence direction towards an output direction.
Clause 99. The ophthalmic laser system according to one of the preceding clauses 87 to 98,
wherein the reflection prism has a first prism side facing the primary beam coming from a first incidence direction, a second prism side facing one or more secondary beams coming from a second incidence direction, and a third prism side facing one or more reflected beams of the one or more secondary beams reflected or back scattered by an eye of a patient along a reflected or back scattered direction.
Clause 100. The ophthalmic laser system according to the preceding clause,
wherein one or more of the first prism side, the second prism side and the third prism side include reflective surfaces comprising at least partial optical coatings for reflecting the primary beam and/or secondary beam.
Clause 101. The ophthalmic laser system according to the preceding clause,
wherein one or more of the first prism side, the second prism side and the third prism side are additionally or alternatively adapted for enabling a total internal reflection of the primary beam and/or the secondary beam.
Clause 102. The ophthalmic laser system according to the preceding clause,
wherein the optical coatings include dichroic, notch and/or partial coatings.
Clause 103. The ophthalmic laser system according to one of the clauses 99 to 102,
wherein the first prism side and the second prism side contact each other at a functional prism angle, wherein the functional prism angle is in the range of 22.5° to 42.5°, preferably of about 25°.
Clause 104. The ophthalmic laser system according to one of the preceding clauses 99 to 103,
wherein the reflection prism adapted to guide the secondary beam along a combined beam path,
wherein the secondary beam is deflected under a secondary beam deflection angle, wherein the secondary beam deflection angle is defined between the second incidence direction and the output direction, wherein the secondary beam deflection angle is in a range of 45° to 85°, preferably of about 50°.
Clause 105. The ophthalmic laser system according to one of the preceding clauses 99 to 104,
wherein the reflection prism is adapted to guide the primary beam along a primary beam path,
wherein the primary beam is deflected under a primary beam deflection angle, wherein the primary beam deflection angle is defined between the first incidence direction and the output direction.
Clause 106. The ophthalmic laser system according to the preceding clause in combination with clauses 103 and 104,
wherein the functional prism angle, the secondary beam deflection angle and the primary beam deflection angle follow the relationships: β=2*α and γ=2*β.
Clause 107. The ophthalmic laser system according to one of the preceding clauses 100 to 106,
wherein the second incidence direction is essentially perpendicular to the second prism side.
Clause 108. The ophthalmic laser system according to one of the preceding clauses 99 to 107,
wherein the reflected or back scattered direction is essentially perpendicular to the third prism side or wherein the reflected or back scattered direction is inclined at an angle of up to 45° with respect to a direction perpendicular to the third prism side.
Clause 109. The ophthalmic laser system according to one of the clauses 99 to 108,
wherein reflection prism is configured to couple the primary beam into reflection prism at the first prism side such that the primary beam travels through the reflection prism towards the second prism side and is then internally reflected at the second prism side towards the third prism side, and
wherein the primary beam is then coupled out of the reflection prism at the third prism side towards the eye.
Clause 110. The ophthalmic laser system according to the preceding clause,
wherein the primary beam is coupled out of the reflection prism essentially perpendicular to the third prism side along the output direction.
Clause 111. The ophthalmic laser system according to one of the clauses 99 to 110,
wherein the first prism side comprises a dichroic mirroring surface covering at least partially the first prism side,
wherein the dichroic mirroring surface is at least partially transparent to the primary beam and at least partially reflective to the secondary beam having one or more different optical properties than the primary beam.
Clause 112. The ophthalmic laser system according to one of the clauses 99 to 111,
wherein the reflection prism is configured to couple the secondary beam into the reflection prism at the second prism side such that the secondary beam travels through the reflection prism towards the first prism side and is reflected at the dichroic mirroring surface of the first prism side towards the second prism side,
wherein the secondary beam then is internally reflected at the second prism side towards the third prism side, and
wherein the secondary beam is coupled out of the reflection prism at the third prism side towards the eye.
Clause 113. The ophthalmic laser system according to the preceding clause,
wherein the outcoupled secondary beam is then at least partially reflected or backscattered by one or more features of the eye along a reflected or back scattered direction being essentially opposite to the output direction,
wherein the reflection prism is configured to couple the reflected or back scattered secondary beam into the reflection prism at the third side such that the reflected or back scattered secondary beam travels through the reflection prism towards the second prism side and is then internally reflected at the second prism side towards the first prism side,
wherein the reflected or back scattered secondary beam is then reflected at the dichroic mirroring surface of the first prism side towards the second prism side, and
wherein the reflected or back scattered secondary beam is then coupled out of the reflection prism at the second prism side towards one or more detectors adapted to detect the reflected or back scattered secondary beam.
Clause 114. The ophthalmic laser system according to the preceding clause,
wherein the reflected or back scattered secondary beam is coupled out of the reflection prism essentially perpendicular to the second prism side.
Clause 115. The ophthalmic laser system according to one of the clauses 99 to 114,
wherein the primary beam is coupled into the reflection prism at the first prism side at an angle in a range of 80° to 100°, preferably 85° to 95°, more preferably 82° to 92°.
Clause 116. The ophthalmic laser system according to one of the clauses 99 to 115,
wherein the primary beam is internally reflected at the second prism side at an angle in a range of larger than 90° to 170°, preferably of about 100°.
Clause 117. The ophthalmic laser system according to one of the clauses 99 to 116,
wherein the secondary beam and/or the reflected or back scattered secondary beam is reflected at the dichroic mirroring surface of the first prism side at an angle in a range of 45° to 85°, preferably of about 50°.
Clause 118. The ophthalmic laser system according to one of the clauses 99 to 117,
wherein the secondary beam and/or the reflected or back scattered secondary beam is internally reflected at the second prism side at an angle in a range of larger than 90° to 170°, preferably of about 100°.
Clause 119. The ophthalmic laser system of one of the preceding clauses 87 to 118,
wherein the beam delivery system is configured for focusing the primary beam so that the primary beam travels toward the eye to be treated.
Clause 120. An ophthalmic laser system configured to perform laser treatments on an eye of a patient using a primary beam, the ophthalmic laser system comprising:
a truncated cone-shaped optical element for guiding a primary beam of the ophthalmic laser system toward the eye,
wherein the truncated cone-shaped optical element is arranged between the eye and a stationary deflection element adapted to deflect the primary beam at a deflection angle toward the eye,
wherein the truncated cone-shaped optical element tapers along a second optical axis towards the eye.
Clause 121. The ophthalmic laser system of the preceding clause,
wherein the ophthalmic laser system comprises an optical measuring system for acquiring measurement data of at least a portion of the eye or for illuminating at least a portion of the eye.
Clause 122. The ophthalmic laser system of the preceding clause,
wherein the stationary deflection element is adapted to combine one or more secondary beams of the optical measuring system with the primary beam, and
wherein the truncated cone-shaped optical element is adapted to guide the combined primary beam and the one or more secondary beams towards the eye.
Clause 123. The ophthalmic laser system of the preceding clause,
wherein the stationary deflection element is a reflection prism, comprising a first prism side facing the primary beam coming from a first incidence direction, a second prism side facing the one or more secondary beams coming from the second incidence direction, and a third prism side facing one or more reflected beams of the one or more secondary beams reflected or backscattered by the eye along a reflected or backscattered direction,
wherein the truncated cone-shaped optical element comprises an essentially flat first cone end surface facing the third prism side and an oppositely arranged second cone end surface facing the eye.
Clause 124. The ophthalmic laser system of the preceding clause,
wherein the reflection prism is a Bauernfeind prism.
Clause 125. The ophthalmic laser system of one of the preceding clauses 123 or 124,
wherein the reflection prism is a beam combining prism configured to combine a beam path of the primary beam and at least one secondary beam path of at least one secondary beam of the optical measuring system.
Clause 126. The ophthalmic laser system of one of the preceding clauses 123 to 125,
wherein the essentially flat first cone end surface contacts at least partially the third prism side.
Clause 127. The ophthalmic laser system of one of the preceding clauses 123 to 126,
further comprising a patient interface arranged between the second cone end surface and the eye for docking the eye to the ophthalmic laser system.
Clause 128. The ophthalmic laser system of one of the preceding clauses 120 to 127,
wherein the truncated cone-shaped optical element is adapted to provide the primary beam to the eye at a numerical aperture of 0.1 to 1.0, preferably of 0.15 to 0.6, more preferably of 0.15 to 0.4 and most preferred of 0.18 to 0.25 is provided.
Clause 129. A method for operating an ophthalmic laser system according to one of the clauses 1 to 86, comprising the steps of:
providing a beam delivery system for focusing the primary beam,
providing a scanning system for scanning a focus of the primary beam within the eye or on an anterior surface of the eye in two or more dimensions,
wherein the beam delivery system comprises a stationary deflection element adapted to deflect the primary beam at a deflection angle toward the eye,
wherein the deflection angle is defined as an angle between:
   an incident portion of the primary beam along a first optical axis of one or more upstream optical elements of the beam delivery system, wherein the upstream optical elements are arranged in a beam path of the primary beam upstream of the stationary deflection element; and
   a deflected portion of the primary beam along a second optical axis of one or more downstream optical elements of the beam delivery system, wherein the downstream optical elements are arranged in the beam path of the primary beam downstream of the stationary deflection element;
deflecting primary beam at angle larger than 90°.
Clause 130: The method according to the preceding clause,
wherein the beam delivery system configured for focusing the primary beam so that the primary beam travels toward the eye to be treated.
Clause 131. A method for operating an ophthalmic laser system according to one of the clauses 88 to 119, comprising the steps of:
providing a beam delivery system for focusing the primary beam,
providing a reflection prism adapted to internally deflect the primary beam toward the eye, and
irradiating the primary beam toward the reflection prism, wherein the primary beam is internally deflected from the reflection prism toward the eye to be treated.
Clause 132. The method according to the preceding clause,
wherein the beam delivery system is configured for focusing the primary beam so that the primary beam travels toward the eye to be treated.
Clause 133. A method for operating an ophthalmic laser system according to one of the clauses 120 to 128, comprising the steps of:
providing a truncated cone-shaped optical element,
arranging the truncated cone-shaped optical element between the eye and a stationary deflection element adapted to deflect the primary beam at a deflection angle toward the eye,
wherein the truncated cone-shaped optical element tapers along a second optical axis towards the eye,
guiding the primary beam of the ophthalmic laser system from the stationary deflection element via the truncated cone-shaped optical element to the eye to be treated.

1. An ophthalmic laser system configured to perform laser treatments on an eye of a patient using a primary beam comprising a treatment laser beam, the ophthalmic laser system comprising a plurality of components including:
a beam delivery system for focusing the primary beam so that the primary beam travels toward the eye to be treated,
a scanning system for scanning a focus of the primary beam within the eye or on an anterior surface of the eye in two or more dimensions,
wherein the beam delivery system comprises a stationary deflection element adapted to deflect the primary beam at a deflection angle toward the eye,
wherein the deflection angle is defined as an angle between:
(a) an incident portion of the primary beam along a first optical axis of one or more upstream optical elements of the beam delivery system, wherein the upstream optical elements are arranged in a beam path of the primary beam upstream of the stationary deflection element; and
(b) a deflected portion of the primary beam along a second optical axis of one or more downstream optical elements of the beam delivery system, wherein the downstream optical elements are arranged in the beam path of the primary beam downstream of the stationary deflection element;
wherein the deflection angle is larger than 90°.

2. The ophthalmic laser system according to claim 1,
wherein the deflection angle provides an increased distance or an increased free space between the patient and one or more of the plurality of components of the ophthalmic laser system compared to a 90° deflection angle.

3. The ophthalmic laser system according to claim 2,
wherein the beam delivery system comprises a patient interface portion comprising a patient interface for docking the eye to the ophthalmic laser system,
wherein the increased distance or the increased free space is provided upstream of the patient interface portion.

4. The ophthalmic laser system according to claim 2,
wherein the beam delivery system comprises a housing,
wherein the housing is adapted to accommodate at least partially one or more of the plurality of components of the ophthalmic laser system,
wherein the housing comprises an inclined portion upstream of the patient interface portion, wherein the inclined portion faces towards the patient,
wherein the increased distance or the increased free space is provided between the patient and the inclined portion of the housing.

5. The ophthalmic laser system according to claim 4,
wherein at least a portion of an exterior housing surface of at least a portion of the inclined portion of the housing faces a portion of the patient, and
wherein the exterior housing surface follows at least partially a shape of the portion of the patient.

6. The ophthalmic laser system according to claim 1,
wherein the ophthalmic laser system comprises an optical measuring system for acquiring measurement data of at least a portion of the eye,
wherein at least one beam of the optical measuring system is combined with the primary beam by the stationary deflection element.

7. The ophthalmic laser system according to claim 1,
wherein the stationary deflection element is part of a first beam combiner configured to combine a beam path of the primary beam and at least one secondary beam path of at least one secondary beam.

8. The ophthalmic laser system according to claim 7,
wherein the secondary beam path comprises one or more beam paths of beams of the optical measuring system.

9. The ophthalmic laser system according to claim 8,
wherein the stationary deflection element comprises a reflection prism for deflecting the primary beam.

10. The ophthalmic laser system according to claim 9,
wherein the reflection prism is further configured for deflecting the secondary beam via one or more internal reflections from a second incidence direction towards an output direction.

11. The ophthalmic laser system according to claim 10,
wherein the stationary deflection element is formed by the reflection prism, wherein the reflection prism is a beam combining prism configured to combine the primary beam and the secondary beam to a common beam path along the output direction.

12. The ophthalmic laser system according to claim 10,
wherein the reflection prism is adapted to reflect the secondary beam internally twice from the second incidence direction towards the output direction.

13. The ophthalmic laser system according to claim 9, wherein the reflection prism is a Bauernfeind prism.

14. The ophthalmic laser system according to claim 9,
wherein the reflection prism has a first prism side facing the primary beam coming from a first incidence direction, a second prism side facing one or more secondary beams coming from the second incidence direction, and a third prism side facing one or more reflected beams of the one or more secondary beams reflected or back scattered by the eye along a reflected or back scattered direction.

15. The ophthalmic laser system according to claim 14,
wherein the reflection prism is configured to couple the primary beam into the reflection prism at the first prism side such that the primary beam travels through the reflection prism towards the second prism side and is then internally reflected at the second prism side towards the third prism side, and
wherein the primary beam is then coupled out of the reflection prism at the third prism side towards the eye.

16. An ophthalmic laser system configured to perform laser treatments on an eye of a patient using a primary beam comprising a treatment laser beam, the ophthalmic laser system comprising:
a beam delivery system for focusing the primary beam so that the primary beam travels toward the eye to be treated,
wherein the beam delivery system comprises a reflection prism adapted to internally deflect the primary beam toward the eye.

17. An ophthalmic laser system configured to perform laser treatments on an eye of a patient using a primary beam, the ophthalmic laser system comprising:
a truncated cone-shaped optical element for guiding a primary beam of the ophthalmic laser system toward the eye,
wherein the truncated cone-shaped optical element is arranged between the eye and a stationary deflection element adapted to deflect the primary beam at a deflection angle toward the eye,
wherein the truncated cone-shaped optical element tapers along a second optical axis towards the eye.

18. A method for operating an ophthalmic laser system according to claim 1, comprising the steps of:
providing a beam delivery system for focusing the primary beam so that the primary beam travels toward the eye to be treated,
providing a scanning system for scanning a focus of the primary beam within the eye or on an anterior surface of the eye in two or more dimensions,
wherein the beam delivery system comprises a stationary deflection element adapted to deflect the primary beam at a deflection angle toward the eye,
wherein the deflection angle is defined as an angle between:
(a) an incident portion of the primary beam along a first optical axis of one or more upstream optical elements of the beam delivery system, wherein the upstream optical elements are arranged in a beam path of the primary beam upstream of the stationary deflection element; and
(b) a deflected portion of the primary beam along a second optical axis of one or more downstream optical elements of the beam delivery system, wherein the downstream optical elements are arranged in the beam path of the primary beam downstream of the stationary deflection element;
deflecting the primary beam at an angle larger than 90°.

19. A method for operating an ophthalmic laser system according to claim 16, comprising the steps of:
providing a beam delivery system for focusing the primary beam so that the primary beam travels toward an eye to be treated,
providing a reflection prism adapted to internally deflect the primary beam toward the eye, and
irradiating the primary beam toward the reflection prism, wherein the primary beam is internally deflected from the reflection prism toward the eye to be treated.

20. A method for operating an ophthalmic laser system according to claim 17, comprising the steps of:
providing a truncated cone-shaped optical element,
arranging the truncated cone-shaped optical element between the eye and a stationary deflection element adapted to deflect the primary beam at a deflection angle toward the eye,
wherein the truncated cone-shaped optical element tapers along a second optical axis towards the eye,
guiding the primary beam of the ophthalmic laser system from the stationary deflection element via the truncated cone-shaped optical element to the eye to be treated.
